(19) United States
(12) Patent Application Publication
(10) Pub. No.: US 2006/0018835 A1
(43) Pub. Date: Jan. 26, 2006

(54) Nanoparticles with inorganic core and methods of using them

(75) Inventors: Patrick Roland Lucien Malenfant, Clifton Park, NY (US); Havva Acar, Clifton Park, NY (US); Peter John Bonitatebus JR., Guilderland, NY (US); William Thomas Dixon, Clifton Park, NY (US); Amit Mohan Kulkarni, Clifton Park, NY (US)

Correspondence Address: GENERAL ELECTRIC COMPANY;GLOBAL RESEARCH, PATENT DOCKET RM. BLDG. K1-4A59, NISKAYUNA, NY, 12309, US

(73) Assignee: General Electric Company

(21) Appl. No.: 10/818,235

(22) Filed: Apr. 2, 2004

(51) Int. Cl. A61K 49/18; A61K 9/16; A61K 9/50

(52) U.S. Cl. 424/9.3; 424/490

(57) ABSTRACT

An aspect of the invention includes a nanoparticle including a substantially monodisperse inorganic core with a surface and a coating substantially covering the surface of the substantially monodisperse inorganic core, wherein the coating includes of at least coating structure I, II, or III wherein the nanoparticle is substantially non-agglomerated and has diameter in a range from about 1 nm to about 100 nm. An aspect of the invention also encompasses a method of making a substantially non-agglomerated nanoparticle having a diameter in a range from about 1 nm to about 100 nm including a substantially monodisperse inorganic core with a surface and a coating substantially covering the surface of the substantially monodisperse inorganic core, wherein the coating comprises coating structure I, II, or III. An aspect of the invention also encompasses various methods of using the substantially non-agglomerated nanoparticle having a diameter in a range from about 1 nm to about 100 nm including a substantially monodisperse inorganic core with a surface and a coating substantially covering the surface of the substantially monodisperse inorganic core, wherein the coating comprises coating structure I, II, or III.

### FIELD OF THE INVENTION

The present invention relates to the preparation of non-agglomerated nanoparticles with an inorganic core via ligand exchange and methods of using them. Particularly, the present invention is directed to novel coatings for nanoparticles and methods of using the nanoparticles in magnetic resonance imaging settings.

### DESCRIPTION OF RELATED ART

Magnetic resonance (MR) imaging is widely used to obtain anatomical images of human subjects for clinical diagnosis. The MR method of imaging is also considered the least invasive method of diagnostic imaging, as it does not expose the patient to potentially harmful high-energy radiation such as X-rays or radioactive isotopes such as technetium-99m. In magnetic resonance imaging (MRI), the image of an organ or tissue is obtained by placing a subject in a strong external magnetic field and observing protons (typically hydrogen nuclei of water) present in the subject's organs or tissues after excitation by a radio frequency magnetic field. The proton relaxation times, termed as T1 (longitudinal relaxation time) and T2 (transverse relaxation time) depend on the chemical and physical environment of the organ or tissue water protons. T1 and T2 vary from tissue to tissue and strongly affect image intensity.

To generate an MR image with good contrast, the T1 and/or T2 of the tissue to be imaged must be different from the background tissue. One way of improving contrast of MR images is to use a MRI contrast agent.

Existing MRI contrast agents, such as paramagnetic metal complexes or superparamagnetic iron oxides, have several disadvantages. For example, although existing paramagnetic contrast agents can reduce T1 and thereby improve contrast, the paramagnetic contrast agents suffer from various disadvantages, such as adverse reactions, short blood circulation times, and potential toxicity. (See Weinmann et al., Am. J. Rad. v.142, 619, 1984; Grief et al. Radiology v.157, 461, 1985; Brasch, Radiology v.147, 781, 1983). For example, many paramagnetic metal complexes are hypertonic and often result in adverse reactions upon injection. Furthermore, the release of the paramagnetic metal ion such as gadolinium, which is highly toxic in the free ionic form, can result in adverse reactions as well. (See “Contrast Media: Biological Effects and Clinical Application”, Vol. I, Ch. 5. Parvez, Z., Moncada, R., and Sovak, M. (Eds.), CRC Press, Boca Raton, Fla. (1987)).

Existing superparamagnetic particle contrast agents also suffer from various disadvantages, such as wide size distribution, agglomeration, and instability. For example, U.S. Pat. No. 4,827,945 describes an aqueous synthesis to prepare superparamagnetic iron oxide particles. Aqueous synthesis results in particles with a wide size distribution, ranging from a few nanometers (nm) a to few microns. This wide, non-uniform size distribution further necessitates extensive de-aggregation, size separation and cleaning steps, which are complicated, time consuming, and expensive. In addition to wide size distribution, another disadvantage is agglomeration. For example, superparamagnetic nanoparticles coated with dextran, dendrimers or liposomes such as described in U.S. Pat. No. 5,219,554 produce agglomerated particles with sizes >100 nm. Due to their large size and surface chemistry, these agglomerated particles are rapidly taken up by macrophages of the reticular endothelial system (RES) upon injection and sequestered by organs such as the liver, spleen and bone marrow. Consequently, these particles have very short blood circulation times and are poor candidates for targeting applications.

In addition to suffering from disadvantages such as wide size distribution, agglomeration, and short blood half-life, some existing coating materials such as dextran need to be used in excess to stabilize and solubilize the magnetic cores. However, the excess dextran or other coating materials need to be (such as U.S. Pat. Nos. 5,492,814 and 5,314,679) removed before clinical use because excess dextran can cause adverse effects on patients, including toxicity (See Briseid, G. et al. Acta Pharmcol. Et. Toxicol. 1980, 47:119-126, Hedin. H. et al. Int. Arch. Allergy and Immunol. 1997, 113:358-359). Consequently a need still exists for coatings of nanoparticles that can stabilize inorganic core particles, and if present in excess, can be tolerated by patients.

Thus substantially non-agglomerated, stable nanoparticles, coatings of such nanoparticles and methods to prepare such substantially non-agglomerated stable nanoparticles are still needed. More specifically, there still remains a need for nanoparticle MRI contrast agents that minimize toxicity or other discomfort to patients, have a suitably long blood circulation life, have substantially uniform size distribution, are substantially non-agglomerated, are stable, and that do not require excessive size selection and purification steps.

### SUMMARY

The purpose and advantages of the present invention will be set forth and apparent from the description of the embodiments that follow, as well as will be learned by practice of the invention. Additional advantages of the invention will be realized and attained by the methods and systems particularly pointed out in the written description and claims hereof, as well as from the drawings.

To achieve these and other advantages in accordance with the purpose of the invention, as embodied and broadly described, an aspect of the invention includes a nanoparticle comprising a substantially monodisperse inorganic core with a surface and a coating substantially covering the surface of the substantially monodisperse inorganic core, wherein the coating comprises of at least one of: 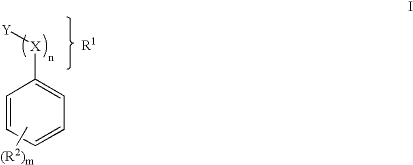
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R independently comprises of at least one of a water-soluble biocompatible polymer; and m is an integer in a range from 1 to about 3; and wherein the nanoparticle is substantially non-agglomerated and has a diameter in a range from about 1 nm to about 100 nm.

An aspect of the invention also encompasses a method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises: 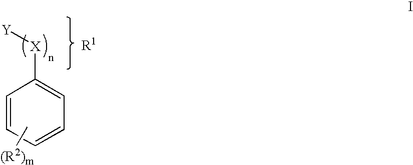
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R independently comprises of at least one of a water-soluble biocompatible polymer; and m is an integer in a range from 1 to about 3; the method comprising: i) contacting the surface of the substantially monodisperse inorganic core with a 1^{st} ligand which is different from the coating structure I; ii) adding a 2 d ligand, wherein the 2 d ligand is the coating structure I, in excess of an amount that is sufficient to replace the 1^{st} ligand; iii) binding the 2^{nd} ligand on the surface of the substantially monodisperse inorganic core; iv) providing an aqueous suspension of the substantially monodisperse inorganic core coated with the 2^{nd} ligand; and v) removing the 1st ligand from the aqueous suspension.

An aspect of the invention also encompasses the composition I described above and its various embodiments.

An aspect of the invention also encompasses other nanoparticles and methods of making them. Another aspect of the invention encompasses a nanoparticle comprising a substantially monodisperse inorganic core with a surface and a coating substantially covering the surface of the substantially monodisperse inorganic core, wherein the coating comprises of at least one of:
Xₙ—R—Si(R¹)₃ II
wherein R independently comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ independently comprises of at least one of an alkoxy, hydroxy, halide, or an alkyl, with the proviso that the three R¹'s cannot all be an alkyl; n is an integer in a range from 1 to about 3; and wherein the nanoparticle is substantially non-agglomerated and has a diameter in a range from about 1 nm to about 100 nm.

Another aspect of the invention encompasses a method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises:
Xₙ—R—Si(R¹)₃ II
wherein R independently comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ independently comprises of at least one of an alkoxy, a hydroxyl, halide, or an alkyl, with the proviso that the three R¹'s cannot all be an alkyl; and n is an integer in a range from 1 to about 3; the method comprising: i) contacting the surface of the substantially monodisperse inorganic core with a 1^{st} ligand which is different from the coating structure II; ii) adding a 2^{nd} ligand, wherein the 2^{nd} ligand is the coating structure II, in excess of an amount that is sufficient to replace the 1^{st} ligand; iii) binding the 2^{nd} ligand on the surface of the substantially monodisperse inorganic core; vi) providing an aqueous suspension of the substantially monodisperse inorganic core coated with the 2^{nd} ligand; v) removing the 1st ligand from the aqueous suspension; and vi) removing some to all of the excess 2^{nd} ligand from the aqueous suspension.

An aspect of the invention also encompasses a nanoparticle comprising a substantially monodisperse inorganic core with a surface and a coating substantially covering the surface of the substantially monodisperse inorganic core, wherein the coating comprises of at least one of:
Xₙ—Y—R—Si(R¹)₃ III
wherein R independently comprises of at least one of an alkyl, an aryl or a combination thereof; R¹ independently comprises of an alkoxy, a hydroxy halide, or an alkyl, with the proviso that the three R¹'s cannot all be an alkyl; n is an integer in a range of 1 to about 3; X comprises of at least one of 0 (zero), H, amino, carboxyl, epoxy, mercapto, cyano, isocyanato, hydroxy, meth(acrylic), or a water-soluble biocompatible polymer and Y comprises 0 (zero) or an organic linkage comprising of at least one of an ether, an thioether, a disulfide, an ester, an amide, a thiourea, an urethane, or a carbamate with the proviso that when X comprises of a water soluble biocompatible polymer, Y comprises 0 or an organic linkage comprising of at least one of an ether, an thioether, a disulfide, an ester, an amide, a thiourea, an urethane, or a carbamate and when X is 0, Y is 0; and wherein the nanoparticle is substantially non-agglomerated and has a diameter in a range from about 1 nm to about 100 nm.

An aspect of the invention also encompasses a method of improving contrast of MR image comprising administering a nanoparticle MRI contrast agent with a coating structure I, II or III to a subject in an amount that is sufficient to differentiate proton relaxation time of a tissue containing the administered nanoparticle MRI contrast agent from a background.

An aspect of the invention also encompasses a magnetic resonance imaging contrast agent in a physiologically acceptable medium, in which the magnetic resonance imaging contrast agent comprises a population of biodegradable superparamagnetic nanoparticles with a coating structure I, II or III, wherein the nanoparticles are capable of being metabolized or excreted by a subject.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with a coating structure I, II or III at a dose in a range from about 0.1 mg to about 100 mg of metal per kg of body weight; and (b) recording the MR image of the tissue or organ of the subject

An aspect of the invention also encompasses a method for obtaining an MR image of the vascular compartment of an animal or a human subject comprising:
(a) administering to the subject, an effective amount of a magnetic resonance imaging contrast agent in a physiologically acceptable medium, wherein the magnetic resonance imaging contrast agent comprises nanoparticles with coating structure I, II or III at a dose in a range from about 0.1 mg to about 100 mg of metal per kg of body weight; and (b) recording the MR image of the vascular compartment.

An aspect of the invention also encompasses a method of diagnosis comprising administering to a mammal a contrast effective amount of nanoparticles with coating structure I, II or III suspended or dispersed in a physiologically tolerable carrier and generating a magnetic resonance image of said mammal.

It is to be understood that both the foregoing general description and the following detailed description are exemplary and are intended to provide further explanation of the invention claimed.

The accompanying drawings, which are incorporated in and constitute part of this specification, are included to illustrate and provide a further understanding of the method and system of the invention. Together with the description, the drawings serve to explain the principles of the invention.

### BRIEF DESCRIPTION OF THE DRAWINGS

FIG. 1 is a ¹H NMR spectrum of a coating structure I as its methyl ester form.

FIG. 2 is a ¹H NMR spectrum of a coating structure I as its carboxylic acid form. Inset is ¹³C-NMR spectrum that corroborates what is seen in the ¹H NMR spectrum.

FIG. 3 is a transmission electron microscopy image of a nanoparticle coated with a coating structure II wherein the coating II is 2-[methoxy(polyethyleneoxy)propyl] trimethoxysilane as described in example 1.

FIG. 4 is a transmission electron microscopy image of a nanoparticle coated with coating structure II wherein the coating II is 2-[methoxy(polyethyleneoxy)propyl] trimethoxysilane as described in example 2.

FIG. 4a is a transmission electron microscopy image of a nanoparticle coated with N-(triethoxysilyl propyl)-N′-(methoxy poly(ethylene glycol))urea wherein poly(ethylene glycol) is 5,000 Da.

FIG. 5 is a transmission electron microscopy image of a nanoparticles with coating structure I wherein R² is PEG-750, Y is COOH, n is 0 and m is 3.

FIG. 6 shows a characteristic magnetization curve as a function of magnetic field for nanoparticles with coating structure I wherein R² is PEG-750, Y is COOH, n is 0 and m is 3, indicating the superparamagnetic nature of the nanoparticles.

FIG. 7A shows a T2 weighted MR image of a mouse before injection of nanoparticles with coating structure I wherein R² is PEG-750, Y is COOH, n is 0, and m is 3.

FIG. 7B shows a T2 weighted MR image of the same mouse 20 minutes after injection of nanoparticles with coating structure I wherein R² is PEG-750, Y is COOH, n is 0, and m is 3.

FIG. 7C shows a T2 weighted MR image of same mouse 24 hours after injection of nanoparticles with coating structure I wherein R² is PEG-750, Y is COOH, n is 0, and m is 3.

FIG. 7D shows the normalized signal intensities of the liver (circled areas in FIGS. 7A, B and C) before injection (A), 20 minutes after injection (B), and 24 hours after injection (C).

FIG. 8A shows T1 weighted images of an inferior vena cava (IVC) of a rat before injection of nanoparticle with coating structure I wherein R² is PEG-750, Y is COOH, n is 0, and m is 3.

FIG. 8B shows T1 weighted images of the same inferior vena cava 10 minutes after injection of nanoparticles with coating structure I wherein R² is PEG-750, Y is COOH, n is 0, and m is 3.

FIG. 8C shows the normalized signal intensities of the IVC (circled areas in FIGS. 8A and B) before injection (A) and 10 minutes after injection (B).

FIG. 9A shows a T2 weighted MR image of a mouse before injection of nanoparticles with coating structure II wherein the coating II is 2-[methoxy(polyethyleneoxy)propyl]trimethoxysilane and m is 6-9.

FIG. 9B shows a T2 weighted MR image of the same mouse 10 minutes after injection of nanoparticles with coating structure II wherein the coating II is 2-[methoxy(polyethyleneoxy)propyl]trimethoxysilane and m is 6-9.

FIG. 9C shows the normalized signal intensities of the liver (circled areas in FIGS. 9A and B) before injection (A) and 10 minutes after injection (B).

FIG. 10A shows T1 weighted images of jugular veins (circled) of a rat before injection of nanoparticles with coating structure II wherein the coating II is 2-[methoxy(polyethyleneoxy)propyl]trimethoxysilane.

FIG. 10B shows T1 weighted images of the same jugular veins (circled) 10 minutes after injection of nanoparticles with coating structure II wherein the coating II is 2-[methoxy(polyethyleneoxy)propyl]trimethoxysilane.

FIG. 10C shows the normalized signal intensities of the jugular veins (circled areas in FIGS. 10A and B) before injection (A) and 10 minutes after injection (B).

### DETAILED DESCRIPTION OF THE PREFERRED EMBODIMENT

Reference will now be made in detail to the preferred embodiments of the invention, examples of which are illustrated below.

An aspect of the invention comprises a nanoparticle comprising a substantially monodisperse inorganic core with a surface and a coating substantially covering the surface of the substantially monodisperse inorganic core, wherein the coating comprises of at least one of: 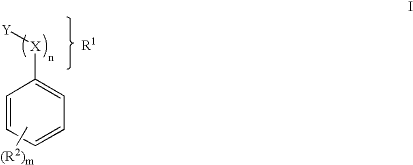
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R² independently comprises of at least one of a water-soluble biocompatible polymer; and m is an integer in a range from 1 to about 3; and wherein the nanoparticle is substantially non-agglomerated and has a diameter in a range from about 1 nm to about 100 nm.

An aspect of the invention also encompasses a method of making a substantially non-agglomerated nanoparticle having a diameter in a range from about 1 nm to about 100 nm comprising a substantially monodisperse inorganic core with a surface and a coating substantially covering the surface of the inorganic core, wherein the coating comprises: 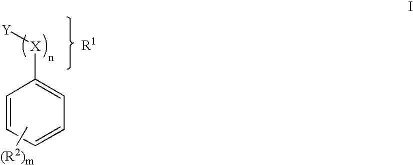
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R² independently comprises of at least one of a water-soluble biocompatible polymer; and m is an integer in a range from 1 to about 3; the method comprising: i) contacting the surface of the substantially monodisperse inorganic core with a 1^{st} ligand which is different from the coating structure I; ii) adding a 2^{nd} ligand, wherein the 2^{nd} ligand is the coating structure I, in excess of an amount that is sufficient to replace the 1^{st} ligand; iii) binding the 2^{nd} ligand on the surface of the substantially monodisperse inorganic core; iv) providing an aqueous suspension of the substantially monodisperse inorganic core coated with the 2^{nd} ligand; and v) removing the 1st ligand from the aqueous suspension.

An aspect of the invention also encompasses the composition I described above and its various embodiments.

An aspect of the invention also encompasses other nanoparticles and methods of making them. Another aspect of the invention encompasses a nanoparticle comprising a substantially monodisperse inorganic core and a coating substantially covering the surface of the substantially monodisperse inorganic core, wherein the coating comprises of at least one of:
Xₙ—R—Si(R¹)₃ II
wherein R independently comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ independently comprises of at least one of an alkoxy, hydroxy, halide, or an alkyl, with the proviso that the three R¹'s cannot all be an alkyl; n is an integer in a range from 1 to about 3; and wherein the nanoparticle is substantially non-agglomerated and has a diameter in a range from about 1 nm to about 100 nm.

Another aspect of the invention encompasses a method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises:
Xₙ—R—Si(R¹)₃ II
wherein R independently comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ independently comprises of at least one of an alkoxy, a hydroxyl, halide, or an alkyl, with the proviso that the three R¹'s cannot all be an alkyl; and n is an integer in a range from 1 to about 3; the method comprising: i) contacting the surface of the substantially monodisperse inorganic core with a 1^{st} ligand which is different from the coating structure II; ii) adding a 2^{nd} ligand, wherein the 2^{nd} ligand is the coating structure II, in excess of an amount that is sufficient to replace the 1^{st} ligand; iii) binding the 2^{nd} ligand on the surface of the substantially monodisperse inorganic core; vi) providing an aqueous suspension of the substantially monodisperse inorganic core coated with the 2^{nd} ligand; v) removing the 1st ligand from the aqueous suspension; and vi) removing some to all of the excess 2^{nd} ligand from the aqueous suspension.

An aspect of the invention also encompasses a nanoparticle comprising a substantially monodisperse inorganic core with a surface and a coating substantially covering the surface of the substantially monodisperse inorganic core, wherein the coating comprises of at least one of:
Xₙ—Y—R—Si(R¹)₃ III
wherein R independently comprises of at least one of an alkyl, an aryl or a combination thereof; R¹ independently comprises of an alkoxy, a hydroxy, halide, or an alkyl, with the proviso that the three R¹'s cannot all be an alkyl; n is an integer in a range of 1 to about 3; X comprises of at least one of 0 (zero), H, amino, carboxyl, epoxy, mercapto, cyano, isocyanato, hydroxy, meth(acrylic), or a water-soluble biocompatible polymer and Y comprises 0 (zero) or an organic linkage comprising of at least one of an ether, an thioether, a disulfide, an ester, an amide, a thiourea, an urethane, or a carbamate with the proviso that when X comprises of a water soluble biocompatible polymer, Y comprises 0 or an organic linkage comprising of at least one of an ether, an thioether, a disulfide, an ester, an amide, a thiourea, an urethane, or a carbamate and when X is 0, Y is 0; and wherein the nanoparticle is substantially non-agglomerated and has a diameter in a range from about 1 nm to about 100 nm.

Regarding nanoparticle with coating structure I, an aspect of the invention encompasses a method of improving contrast of MR image comprising administering a nanoparticle MRI contrast agent with a coating structure I to a subject in an amount that is sufficient to differentiate proton relaxation time of a tissue containing the administered nanoparticle MRI contrast agent from a background.

An aspect of the invention also encompasses a magnetic resonance imaging contrast agent in a physiologically acceptable medium, in which the magnetic resonance imaging contrast agent comprises a population of biodegradable superparamagnetic nanoparticles with a coating structure I, wherein the nanoparticles are capable of being metabolized or excreted by a subject.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises the nanoparticle with a coating structure I at a dose in a range from about 0.1 mg to about 100 mg of metal per kg of body weight; and (b) recording the MR image of the tissue or organ of the subject.

An aspect of the invention also encompasses a method for obtaining an MR image of the vascular compartment of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure I at a dose in a range from about 0.1 mg to about 100 mg of metal per kg of body weight; and (b) recording the MR image of the vascular compartment.

An aspect of the invention also encompasses a method of diagnosis comprising administering to a mammal a contrast effective amount of nanoparticles with coating structure I suspended or dispersed in a physiologically tolerable carrier and generating a magnetic resonance image of said mammal.

Regarding nanoparticle with coating structure II, an aspect of the invention encompasses a method of improving contrast of MR image comprising administering a nanoparticle MRI contrast agent with a coating structure II to a subject in an amount that is sufficient to differentiate proton relaxation time of a tissue containing the administered nanoparticle MRI contrast agent from a background.

An aspect of the invention also encompasses a magnetic resonance imaging contrast agent in a physiologically acceptable medium, in which the magnetic resonance imaging contrast agent comprises a population of biodegradable superparamagnetic nanoparticles with coating structure II, wherein the nanoparticles are capable of being metabolized or excreted by a subject.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure II at a dose in a range from about 0.1 mg to about 100 mg of metal per kg of body weight; (b) recording the MR image of the tissue or organ of the subject

An aspect of the invention also encompasses a method for obtaining an MR image of the vascular compartment of an animal or a human subject comprising (a) administering to the subject, an effective amount of a magnetic resonance imaging contrast agent in a physiologically acceptable medium, wherein the magnetic resonance imaging contrast agent comprises nanoparticles with coating structure II at a dose in a range from about 0.1 mg to about 100 mg of metal per kg of body weight; and (b) recording the MR image of the vascular compartment

An aspect of the invention also encompasses a method of diagnosis comprising administering to a mammal a contrast effective amount of nanoparticles with coating structure II suspended or dispersed in a physiologically tolerable carrier and generating a magnetic resonance image of said mammal.

Regarding nanoparticle with coating structure III, an aspect of the invention also encompasses a method of improving contrast of MR image comprising administering a nanoparticle MRI contrast agent with a coating structure III to a subject in an amount that is sufficient to differentiate proton relaxation time of a tissue containing the administered nanoparticle MRI contrast agent from a background.

An aspect of the invention also encompasses a magnetic resonance imaging contrast agent in a physiologically acceptable medium, in which the magnetic resonance imaging contrast agent comprises a population of biodegradable superparamagnetic nanoparticles with coating structure III, wherein the nanoparticles are capable of being metabolized or excreted by a subject.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure III at a dose in a range from about 0.1 mg to about 100 mg of metal per kg of body weight; (b) recording the MR image of the tissue or organ of the subject

An aspect of the invention also encompasses a method for obtaining an MR image of the vascular compartment of an animal or a human subject comprising (a) administering to the subject, an effective amount of a magnetic resonance imaging contrast agent in a physiologically acceptable medium, wherein the magnetic resonance imaging contrast agent comprises nanoparticles with coating structure III at a dose in a range from about 0.1 mg to about 100 mg of metal per kg of body weight; and (b) recording the MR image of the vascular compartment

An aspect of the invention also encompasses a method of diagnosis comprising administering to a mammal a contrast effective amount of nanoparticles with coating structure III suspended or dispersed in a physiologically tolerable carrier and generating a magnetic resonance image of said mammal.

The nanoparticles with coating structure I, II, or III may have chiral centers and occur as racemic mixtures, as individual diastereomers, or as enantiomers with all isomeric forms. The scope of the present invention includes individual enantiomers of compounds of coatings (I), (II), or (III) as well as mixtures of enantiomers of compounds of coatings (I), (II), or (III) in any proportion, including racemic mixtures.

When any variable occurs more than one time in any constituent or in formula (I), (II), and (III) its definition on each occurrence is independent of its definition at every other occurrence. Also, combinations of substituents and/or variables are permissible only if such combinations result in stable compounds.

Some abbreviations that may appear in this application are as follows:

Abbreviations

Unless otherwise noted, substantially non-agglomerated nanoparticle means nanoparticle wherein the diameter is less than 100 nm.

Unless otherwise noted, substantially monodisperse inorganic core means a standard deviation of up to 10%.

Unless otherwise noted, water-soluble polymer includes polyethylene glycol (PEG), a polypropylene glycol (PPG), a poly(N-isopropylacrylamide) (PNIPA), a poly(2-hydroxyethyl) methacrylate (HEMA), a poly vinyl alcohol (PVA), a peptide, a protein, a polysaccharide, or combinations thereof.

Unless otherwise noted, the term “alkyl” includes both branched- and straight chain saturated aliphatic hydrocarbon groups having the specified number of carbon atoms. For example, “C₁₋₆ alkyl” means an alkyl group having 1 to 6 carbon atoms, e.g., 1, 2, 3, 4, 5 or 6. For illustration and not limitation, the alkyl may be methyl, ethyl, propyl, butyl, etc. The alkyl group may be unsubstituted or substituted.

Unless otherwise noted, “halide”, as used herein, includes fluorine, chlorine, bromine, and iodine.

Unless otherwise noted, “alkoxy” means a linear or branched alkyl group of indicated number of carbon atoms attached through an oxygen bridge. For illustration and not limitation, “C₁-6 alkoxy” means any alkoxy having 1 to 6 carbon atoms, e.g., 1, 2, 3, 4, 5 or 6.

Unless otherwise noted, the term “aryl” includes a 6- to 10-membered mono- or bicyclic ring system such as phenyl, or naphthyl. The aryl ring can be unsubstituted or substituted with, for illustration and not limitation, one or more of C₁₋₆ alkyl; C₁₋₆ alkoxy; halogen; or amino.

Unless otherwise noted, the term “solvent” includes any polar and non polar and organic solvents such as, for illustration and not limitation, water, triethylamine, pyridine, isopropyl alcohol, ethanol, methanol, N-methylpyrrolidinone, dimethylformamide, acetonitrile, toluene and tetrahydrofuran.

Unless otherwise noted, the term “binding” includes, for illustration and not limitation, chemisorption and/or physisorption of the coating on the substantially monodisperse inorganic core and/or covalent bonding of the coating to the substantially monodisperse inorganic core.

Administration of nanoparticles comprising a coating structure of formula I, II, or III includes, for illustration and not limitation, orally, topically, parenterally, by inhalation spray or rectally in dosage unit formulations containing conventional non-toxic pharmaceutically acceptable carriers, adjuvants and vehicles. The term parenteral as used herein includes subcutaneous injections, intravenous, intramuscular, intrasternal injection or infusion techniques.

Unless otherwise noted, diameters of nanoparticles were measured by light scattering. Use of the word diameter does not restrict the nanoparticles to spherical shapes.

I. Nanoparticles with Coating Structure I

An aspect of the invention encompasses all variations of the novel nanoparticle comprising a substantially monodisperse inorganic core and a coating substantially covering the surface of the substantially monodisperse inorganic core, wherein the coating comprises of at least one of: 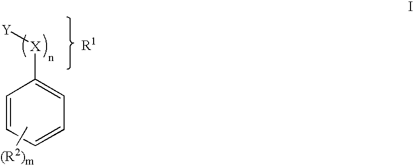
wherein R¹ is X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R² independently comprises of at least one of a water-soluble polymer; and m is an integer in a range from 1 to about 3; and wherein the nanoparticle is substantially non agglomerated and has a diameter in a range from about 1 nm to about 100 nm.

In one aspect, the nanoparticle comprises a coating structure I wherein the coating structure I comprises of at least one of: 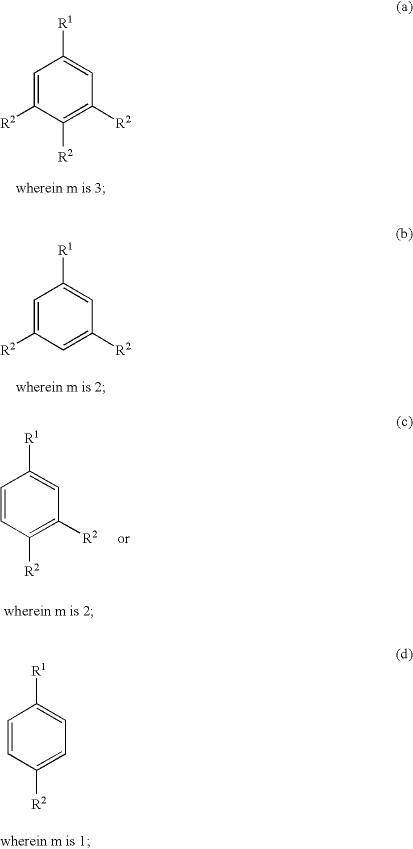
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, and a NH₂; wherein R is a methyl or an ethyl; and R² independently comprises of at least one of a water-soluble biocompatible polymer. In another aspect, the nanoparticle comprises a coating structure I wherein the coating structure I comprises of at least one of: 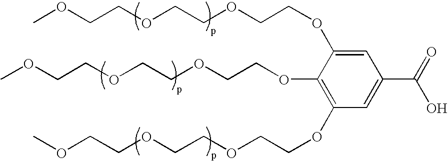
wherein m is 3; Y is COOH; X is O; n is O; R² is 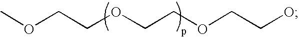
and p is an integer in a range from 5 to about 125. The nanoparticle may be less than 50 nm or less than 25 nm. Furthermore, the R² group of water-soluble biocompatible polymer may comprise of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof. Furthermore, the nanoparticle may comprise a coating where the coating comprises a plurality of variations of the coating structure I.
Synthesis of a Nanoparticle with a Coating of the General Formula I Wherein the Coating I is a PEG Ligand Coating and Synthesis of PEG Coating.

For illustration and not limitation, the following example demonstrates the novel nanoparticle, where the coating I comprises of at least one of: 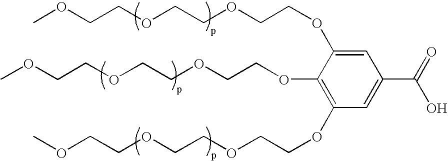
wherein m is 3; Y is COOH; X is O; n is O; R² is 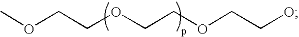
and p is an integer in a range from 5 to about 125.

Ligand/coating synthesis. For illustration and not limitation, the example describes a nanoparticle with the coating of general formula I where the coating I comprises R² as PEG. The invention encompasses the R² groups of coating I to independently be any other designated R² group at various designated locations.

Scheme 1 below depicts the synthesis of the general architecture of a novel R PEG based ligand coating I, triethylene glycol derivative. In this case, 3,4,5 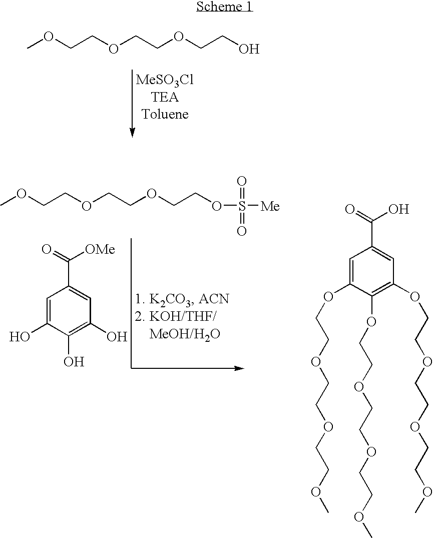
trihydroxy benzoic acid and three R² PEG chains, of the same length were attached to provide a branched ligand with PEGs of the same length. Varying PEG lengths can be attached to provide a branched ligand with PEGs of varying lengths. The branched PEG ligand was chosen to mimic other small molecule ligands that have successfully stabilized nanoparticles such as TOPO (trioctyl phosphine oxide). The PEG framework resists protein adsorption, even at relatively low degrees of polymerization. The carboxylic functionality binds to the surface of the substantially monodisperse inorganic iron oxide core.

The first step (in scheme 1) of preparing a coating I structure with R² as PEG involved preparing PEGs having methane sulfonyl esters, at one end of the polymer chain. Methane sulfonyl esters of PEG were prepared in essentially quantitative yield by reacting methane sulfonyl chloride with the PEG alcohol in toluene in the presence of triethylamine. Methane sulfonated PEGs were used without purification. In the second step, functionalization of 3,4,5 trihydroxy methyl benzoate with three PEG chains was accomplished using standard phase transfer catalyzed conditions. For example, reacting the phenol with PEG methane sulfonates in acetonitrile in the presence of potassium carbonate yields the tris functionalized 3,4,5 (tris PEG) methyl benzoate. The last step was to convert the ester function into a carboxylic acid by simply reacting the 3,4,5 (tris PEG) methyl benzoate with potassium hydroxide in a water/THF/MeOH mixture. Upon acidification, the desired carboxylic is obtained.

Although Scheme 1 depicts the synthesis of the 3,4,5 triethylene glycol derivative PEG 350, 550 and 750 derivatives, described below, were all prepared using the general procedure of scheme 1.
3,4,5 (tris polyethylene glycol) benzoic acid (referred to as PEG-165) Molecular Weight (MW) 611 Da
3,4,5 (tris polyethylene glycol) benzoic acid (referred to as PEG-350) MW 1136 Da
3,4,5 (tris polyethylene glycol) benzoic acid (referred to as PEG-550) MW 1720 Da
3,4,5 (tris polyethylene glycol) benzoic acid (referred to as PEG-750) 2366 Da

Nanoparticle synthesis. An aspect of the invention also encompasses a method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises: 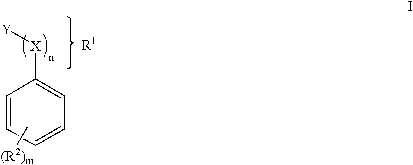
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R² independently comprises of at least one of a water-soluble biocompatible polymer; and m is an integer in a range from 1 to about 3; the method comprising: i) contacting the surface of the substantially monodisperse inorganic core with a 1^{st} ligand which is different from the coating structure I; ii) adding a 2^{nd} ligand, wherein the 2^{nd} ligand is the coating structure I, in excess of an amount that is sufficient to replace the 1^{st} ligand; iii) binding the 2^{nd} ligand on the surface of the substantially monodisperse inorganic core; iv) providing an aqueous suspension of the substantially monodisperse inorganic core coated with the 2^{nd} ligand; and v) removing the 1st ligand from the aqueous suspension.

One aspect of the method is when the 2^{nd} ligand comprises of at least one of following coating structure I: 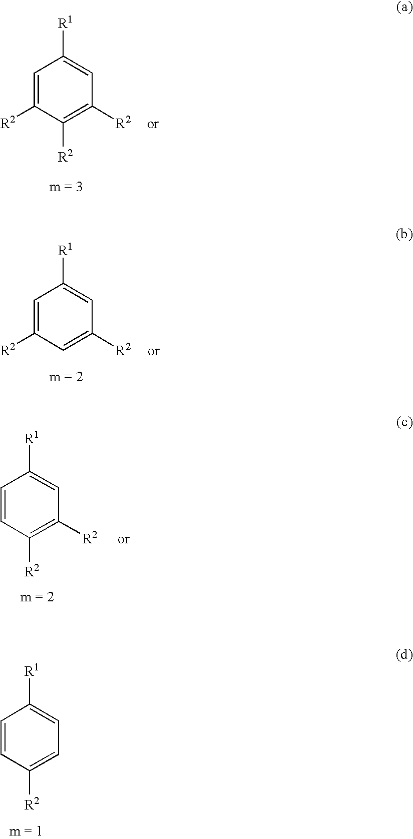
wherein R¹ is (X)ₙ—Y; X is CH₂; n is 0, 1, 2; Y comprises of at least one of COOH, SO₃H, PO₄H, Si(OR)₃, SiCl₃, or NH₂; wherein R is a methyl or an ethyl; R² independently comprises of at least one of water-soluble biocompatible polymer, such as polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof; and m is an integer in a range of from 1 to about 3. Another aspect of the method is when the 2^{nd} ligand comprises of at least one of the following coating structure I: 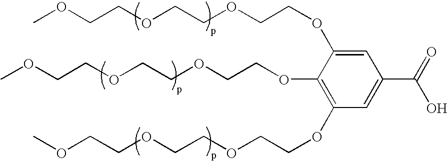
wherein m is 3; Y is COOH; X is O; n is O; R² is 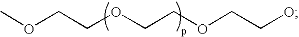
and p is an integer in a range from 5 to about 125.

For illustration and not limitation, Scheme 2 demonstrates the synthesis of a novel nanoparticle with a coating structure I where the coating I comprises of at least one: 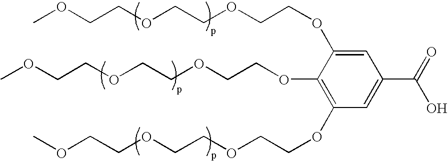
wherein m is 3; Y is COOH; X is O; n is O; R² is 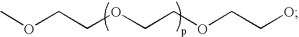
and p is an integer in a range from 5 to about 125.

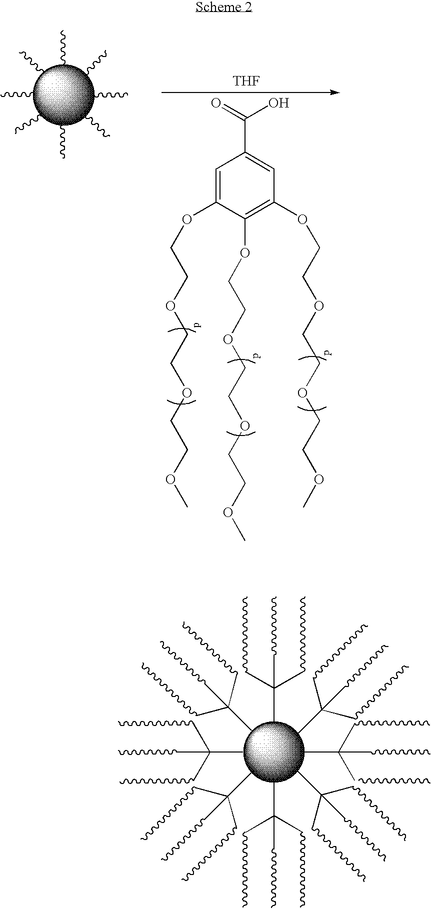

In scheme 2 above, the number of coating structures I around the substantially monodisperse inorganic core was merely for illustration. The number of coating structures I around the substantially monodisperse inorganic core may vary depending on the size of the substantially monodisperse inorganic core and the size of the particular coating structure I. Although the substantially monodisperse inorganic core in Scheme 2 was depicted as being surrounded by the same variation of coating structure I, the nanoparticles with coating I may comprise a plurality of variations of the coating structure I. For example, the substantially monodisperse inorganic core may be surrounded by different variations of coating structures I, as depicted above.

In the above examples, the number of PEG chains for R² was 3 merely for illustration. The invention encompasses the number of PEG chains to be 1-3 at various locations and of varying length. The number, the type, and the location of the PEG chains may vary independently and are within the scope of invention. Another embodiment of the nanoparticles with coating I may be wherein the R² water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

For illustration and not limitation, the nanoparticles comprising coating structure I has been described with R² as PEG. The R² variables of coating composition I may independently be any other designated R² group. For example, R² may be any water-soluble biocompatible polymer. Furthermore, R¹, X, Y, R², n, and m groups of general coating formula I may be any designated R¹, X, Y, R², n, and m groups, respectively, independent of each other. For example, when R¹ is X—COOH, R² may be any designated water-soluble biocompatible polymer. Similarly, when R² is a designated water-soluble biocompatible polymer such as PEG, R¹ may be X—COOH or any other designated R¹ group.

Furthermore, the nanoparticle of (I) may be in the form of a purified single enantiomer, (S) or (R) isomer, or both. The number of molecules that make up the coating around the substantially monodisperse inorganic core may vary depending on the size of the core and the particular coating structure I.

One embodiment of the nanoparticles with coating I has diameter of less than 50 nm. Another embodiment of the nanoparticles with coating I has diameter of less than 25 nm. Another embodiment of the nanoparticles with coating I may be wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof. Furthermore, the nanoparticles with coating I may comprise a plurality of variations of the coating structure I.

The following also illustrates embodiments of synthesis of the nanoparticle. Synthesis of Hydrocarbon-Soluble γ—Fe₂O₃ Nanoparticles. Relatively monodisperse 6 nm iron oxide crystallites (σ<10%) were prepared by rapid injection of Fe(CO)₅ (200 mL, 1.52 mmol) into hot dioctyl ether (8 mL) under nitrogen, containing lauric acid (0.91 g, 4.56 mmol) and trimethylamine N-oxide (0.57 g, 7.60 mmol), followed by a heat treatment procedure. The substantially monodisperse inorganic core γ—Fe₂O₃, is described in U.S. patent application Ser. No. 10/208,046 which is incorporated by reference. Prior to injection, the solvent-oxidant-surfactant mixture was brought to 100° C. under a blanket of nitrogen. Upon injection the temperature increased to 120° C., at which it was kept for 1 h while stirring vigorously. A brown-black solution containing nanoparticles resulted after stirring for another 1 h at reflux (˜290° C.). The flask was allowed to cool, and while stirring continued, acetonitrile was added to deposit a brown-black precipitate (˜20 mL) and excess surfactant. Centrifugation separated solids from supernatant. The resulting golden-brown powder may be solubilized in hydrocarbon solvents, such as heptane and toluene.

Water-Soluble γ—Fe₂O₃ Nanoparticles. Lauric acid coated particles were combined with an 8-fold excess (by mass) of PEG ligand (tris-(3,4,5-PEG-750) benzoic acid) and the solids were solubilized in THF. The homogeneous reaction mixture was allowed to stir overnight at room temperature to ensure complete exchange of surface ligands. The THF solution was diluted with an equivalent volume of water and the THF removed by rotoevaporation. Ligand exchange provided a dark black-brown cloudy solution, with suspended lauric acid crystals. Extraction of the aqueous solution with hexanes effectively removed all the lauric acid. The aqueous solution was then diluted with an equivalent volume of acetone and a transparent solution was obtained. Removal of the acetone by rotoevaporation yielded an aqueous solution of γ—Fe₂O₃ nanoparticles. The aqueous suspensions were filtered through 100 nm filters. The diameter was measured by dynamic light scattering to be 25 nm.

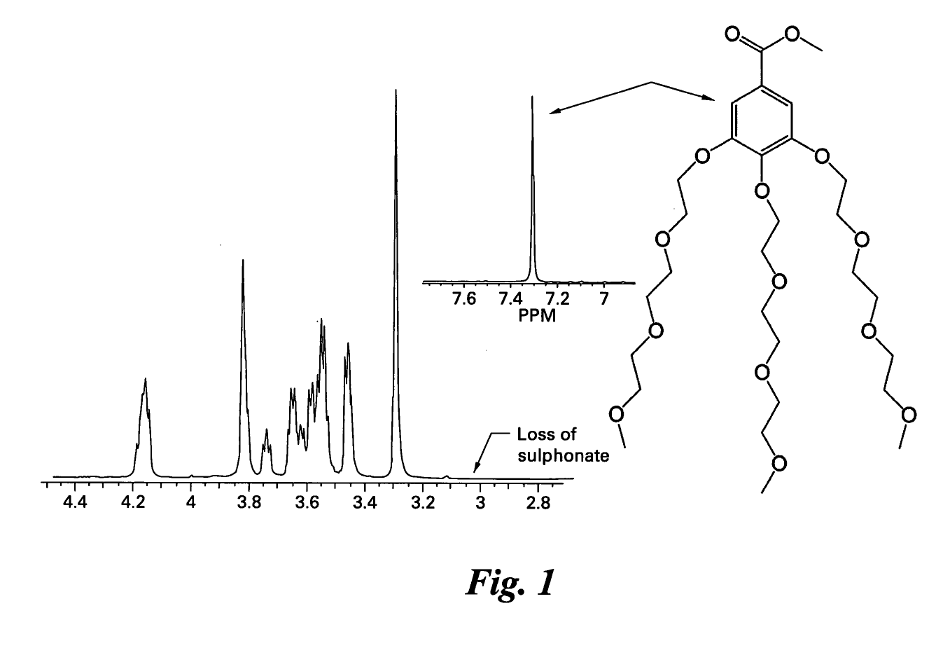

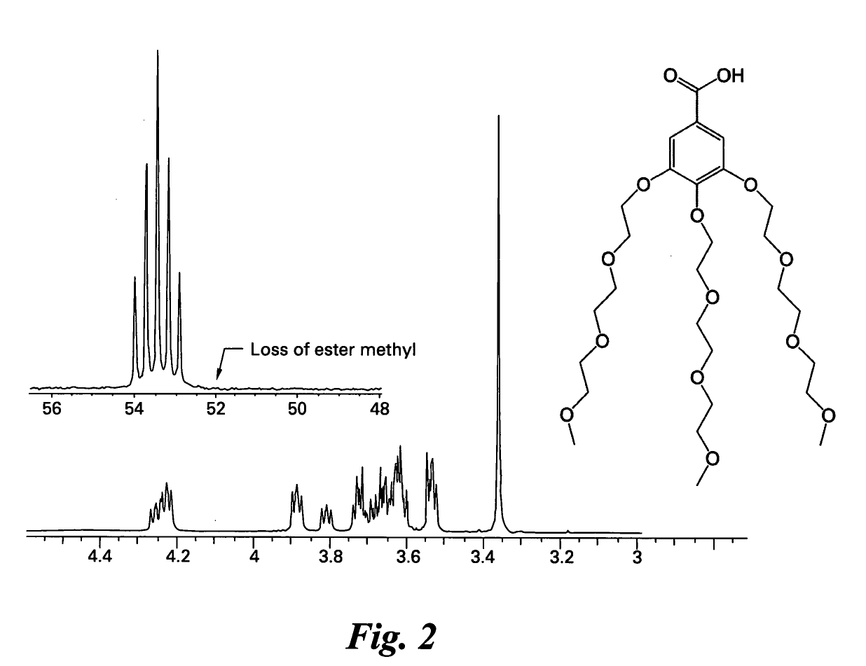

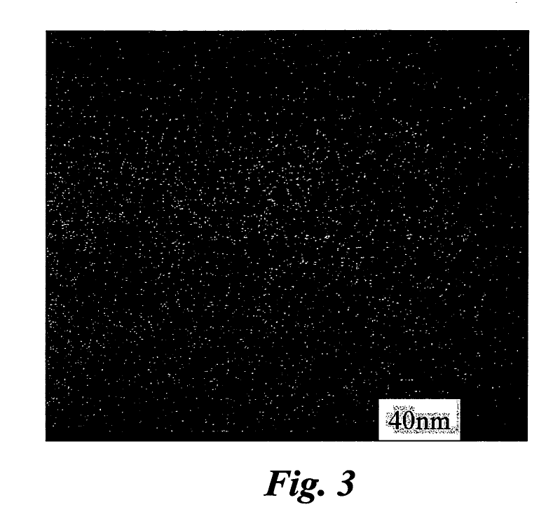

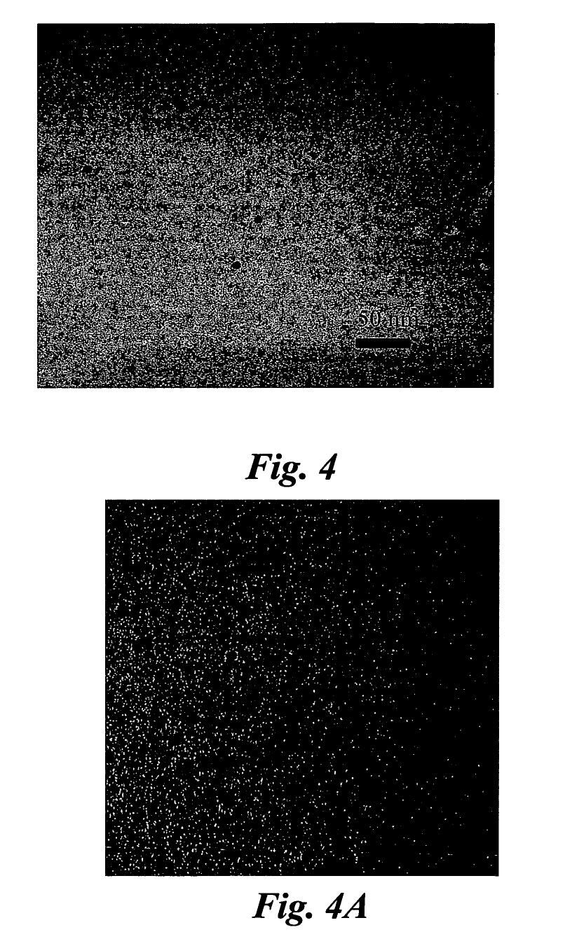

Ligand Characterization. The PEG series of ligands were characterized by ¹H, ¹³C NMR. The Triethylene glycol derivative can be used to provide a general analysis for this class of materials. As can be seen in FIG. 1, the extent of PEG functionalization can be determined by monitoring the aromatic protons at approx. 7.37 ppm. A singlet is expected due to the symmetry of the molecule. α protons to the phenoxy groups are observed at 4.1-4.2 ppm while the absence of excess methyl sulfonyl ester PEG is clearly seen by the lack of any resonance near 3 ppm. Loss of the methyl ester after saponification is seen by the absence of resonance peaks at 50.5 in the ¹³C and 3.83 ppm in the ¹H NMR spectra (FIG. 2). Conventional ei-ms was used to confirm the molecular weight of the triethylene glycol derivative, yet molecular weights for the higher MW materials were confirmed by MALDI-TOF spectrometry.

Characterization

PEG-165 methyl sulfonate. PEG-165-OH (50.0 g; 305 mmol) was charged into a round bottom flask and dissolved in 305 ml of toluene. TEA (32.33 g; 320 mmol) was added and the solution was cooled with an ice-water bath to ca. 0° C. Methyl sulfonyl chloride (36.66 g; 320 mmol) was added dropwise. The reaction was stirred for 1 h, filtered and toluene was removed by rotoevaporation. Trace amounts of toluene were removed under high vacuum distillation condition to provide the desired product as a golden colored oil (73.4 g; 303 mmol; 95%). ¹H NMR (400 MHz, CD₂Cl₂) δ 4.35 (m, 2H), 3.8-3.4 (m, 10H), 3.32 (s, 3H), 3.05 (s, 3H). ¹³C NMR (100 MHz, CD₂Cl₂) 71.8, 70.5, 70.3, 69.7, 68.9, 58.4, 37.3.

Tris (3,4,5 PEG-165) methyl benzoate. PEG-165-mesylate (21.00 g; 87 mmol) was charged into a round bottom flask and dissolved in 110 ml of ACN. K₂CO₃ (10 g) was added, followed by 3,4,5 trihydroxy methyl benzoate (5.0 g; 27 mmol) and the solution was heated to reflux. The reaction was stirred for 2 days. The reaction mixture was cooled to room temperature, filtered and the crude product was purified by flash chromatography (5-10% MeOH in DCM) to provide the desired product as a golden colored oil (84 g; 23 mmol; 85%). ¹H NMR (400 MHz, CD₂Cl₂) δ 7.3 (s, 2H), 4.20 (m, 6H), 3.9-3.4 (m, 33H), 3.33 (s, 9H). ¹³C NMR (100 MHz, CD₂Cl₂) 166.2, 152.3, 142.4, 125.0, 108.7, 72.4, 71.9, 70.7, 70.6, 70.5, 70.45, 70.38, 69.6, 68.85, 58.5, 51.9. MS (FAB+) m/z calcd for (MH)⁺ (C₂₉H₅₀O₁₄) 622.32; found 622.

Tris (3,4,5 PEG-165) benzoic acid. Tris (3,4,5 PEG-165) methyl benzoate (13.4 g; 20 mmol) was charged into a round bottom flask and dissolved in 115 ml of water-MeOH (20:80). KOH (10 g) was added, and the solution was stirred at RT overnight. The reaction mixture was acidified to pH 2 with concentrated HCl, MeOH was removed by rotoevaporation and the aqueous solution was extracted 4× with DCM. The combined organic layers were dried over MgSO₄, filtered and dried in vacuo at 100° C. The desired product was isolated as a golden colored oil (12.9 g; 19.8 mmol; 99%). ¹H NMR (400 MHz, CD₂Cl₂) δ 7.4 (s, 2H), 4.25 (m, 6H), 3.9-3.4 (m, 30H), 3.36 (s, 9H). ¹³C NMR (100 MHz, CD₂Cl₂) 170.0, 152.3, 142.9, 124.4, 109.1, 72.5, 71.9, 70.7, 70.6, 70.5, 70.46, 70.4, 69.6, 68.8, 58.5. MS (FAB−) m/z calcd for (M−H)⁻ (C₂₈H₄₈O₁₄-1H) 607.7, found 607.

An aspect of the invention also encompasses all variations of the novel coating wherein the coating comprises of at least one of: 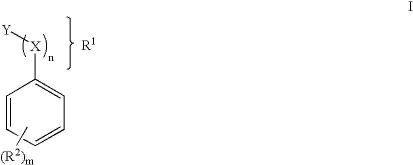
wherein R¹ is (X)ₙ—Y; X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; R² independently comprises of at least one of a water-soluble biocompatible polymer; and m is an integer in a range from 1 to about 3; and wherein the nanoparticle is substantially non agglomerated and has a diameter in a range from about 1 nm to about 100 nm.
II. Nanoparticles With Coating Structure II and III

An aspect of the invention also encompasses all variations of the novel nanoparticle comprising a substantially monodisperse inorganic core and a coating substantially covering the surface of the substantially monodisperse inorganic core wherein the coating comprises of least one of:
Xₙ—R—Si(R¹)₃ II
wherein R comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ comprises of at least one of an alkoxy, a hydroxyl, halide, or an alkyl, with the proviso that the three R¹'s cannot all be an alkyl; n is an integer in a range from 1 to about 3; and wherein the nanoparticle is substantially non-agglomerated and has a diameter in a range from about 1 nm to about 100 nm.

Synthesis of a nanoparticle comprising a coating structure II wherein the coating II comprises of at least one of 2-[methoxy(polyethyleneoxy)propyl]trimethoxysilane.

For illustration and not limitation, the following examples demonstrate novel nanoparticle comprising coating II, where the coating II comprises of at least one of:
CH₃O(CH₂CH₂O)ₘCH₂CH₂CH₂Si(R¹)₃
wherein R¹ is OCH₃ or OCH₂CH₃; R is propyl; n is 1; X is CH₃O(CH₂CH₂O)ₘ; and m is an integer in a range from about 5 to about 115; and wherein the nanoparticle is substantially non-agglomerated and has a diameter in a range from about 1 nm to about 100 nm. More specifically, the following examples demonstrate the following coating structure II:
2-[methoxy(polyethyleneoxy)propyl]trimethoxysilane II
wherein R¹ is OCH₃. Even more specifically, the following examples demonstrate the following coating structure II:
CH₃O(CH₂CH₂O)₆₋₉CH₂CH₂CH₂Si(OCH₃)₃
wherein R¹ is OCH₃ and m is an integer in a range from 6 to about 9.

In the above example, R was propyl for illustration, not limitation. R may comprise of at least one of an alkyl, an aryl or a combination. R¹ was OCH₃ or OCH₂CH₃ for illustration, not limitation. R¹ may comprise of at least one of an alkoxy, a hydroxyl, halide, or an alkyl, with the proviso that the three R¹'s cannot all be an alkyl. X was CH₃O(CH₂CH₂O)ₘ for illustration and not limitation. X may independently comprise of at least one of H, amino, carboxyl, epoxy, mercapto, cyano, isocyanato, hydroxy, meth(acrylic), and a water-soluble biocompatible polymer. Furthermore, the number of X may vary as designated by n where n is an integer in a range from 1 to about 3. Each X may vary independently and are within the scope of invention.

For illustration and not limitation, the nanoparticle with a coating structure II has been described wherein R¹ is OCH₃ or OCH₂CH₃; R is propyl; n is 1; X is CH₃O(CH₂CH₂O)ₘ wherein m is an integer in a range from about 5 to about 115. The R¹, R, X, n, and m variables of the nanoparticle with coating composition II may independently be any designated variable regardless what the other R¹, R, X, n, and m groups may be. For example, X may independently comprise of at least one of H, amino, carboxyl, epoxy, mercapto, cyano, isocyanato, hydroxy, meth(acrylic), and a water-soluble biocompatible polymer regardless of what R¹, R, n, and m variables may be. Similarly, R¹ may independently comprise of at least one of OCH₃ or OCH₂CH₃ regardless of what X, R, n, and m variables may be.

An aspect of the invention also encompasses a method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises:
Xₙ—R—Si(R¹)₃ II
wherein R comprises of at least one of an alkyl, an aryl or a combination thereof; X independently comprises of at least one of H, amino, carboxyl, epoxy, mercapto, cyano, isocyanato, hydroxy, meth(acrylic), or a water-soluble biocompatible polymer; R¹ independently comprises of at least one of an alkoxy, a hydroxyl, halide, or an alkyl, with the proviso that the three R¹'s cannot all be an alkyl; and n is an integer in a range from 1 to about 3; the method comprising: i) contacting the surface of the substantially monodisperse inorganic core with a 1^{st} ligand which is different from the coating structure II; ii) adding a 2^{nd} ligand, wherein the 2^{nd} ligand is the coating structure II, in excess of an amount that is sufficient to replace the 1^{st} ligand; iii) binding the 2^{nd} ligand on the surface of the substantially monodisperse inorganic core; vi) providing an aqueous suspension of the substantially monodisperse inorganic core coated with the 2^{nd} ligand; v) removing the 1st ligand from the aqueous suspension; and vi) removing some to all of the excess 2^{nd} ligand from the aqueous suspension.

Exchange of a first ligand, such as for example, lauric acid, with the second ligand comprising coating structure II, such as for example; 2-[methoxy(polyethyleneoxy)propyl]trimethoxysilane, can be done in solution (toluene, alcohols, etc) or neat in the absence of a solvent. Ligand exchange reaction is followed by condensation of the alkoxy silane group which induces improved stability to the coated particles. Water-soluble particles of typically 10-20 nm can be obtained from PEGSi through this method without further size separation. These particles can be purified through ultrafiltration or centrifugation and sterilized through syringe filtration and injected IV to rats and mouse for MR imaging.

### EXAMPLE 1

### Nanoparticle with Coating II Comprising 2[methoxy(polyethyleneoxy)propyl]trimethoxysilane without solvent

Lauric acid coated (γ—Fe₂O₃) (0.0341 mmol Fe) was mixed with 2.806 g 2-[methoxy(polyethyleneoxy)propyl]trimethoxysilane (Gelest, inc, molecular weight 460-590 g/mol) and sonicated 20 min at RT, then stirred overnight. The substantially monodisperse inorganic core γ—Fe₂O₃, is described in U.S. patent application Ser. No. 10/208,046 which is incorporated by reference. Isopropanol (5 mL) and 0.2 mL NH₄OH (38%) was added to the mixture and sonicated at 55° C. for 6 h, then stirred at RT overnight. Isopropanol was removed by rotary evaporator and the residue was resuspended in 5 mL milliQ water. Nice yellow suspension and white crystals appeared in water. White crystals (lauric acid) were extracted with hexane (3 times 6 mL hexanes wash). Aqueous suspension was filtered through 100 nm filters and the diameter was measured by DLS to be 10 nm.

### EXAMPLE 2

### Nanoparticle with Coating II Comprising 2[methoxy(polyethyleneoxy)propyl]trimethoxysilane with solvent:

Alternatively this ligand exchange was performed in non-protic solvents such as toluene, or protic solvents such as EtOH. As an example, lauric acid coated γ—Fe₂O₃ (0.0129 mmol Fe) was mixed with 20 mg 2-[methoxy(polyethyleneoxy)propyl]trimethoxysilane (Gelest Inc., molecular weight 460-590 g/mol) in 5 mL toluene and sonicated 20 min at RT. Transparent brown suspension was stirred overnight. 100 mL NH₄OH (38%) was added and sonicated at 55° C. for 6 h, then stirred at RT overnight. Toluene was removed by rotary evaporator and the residue was resuspended in 5 mL milliQ water. Aqueous suspension was filtered through 100 nm filters and the diameter was measured by DLS to be 13 nm.

For illustration and not limitation, the above examples of the method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core structure II were demonstrated with coating II comprising:
CH₃O(CH₂CH₂O)ₘCH₂CH₂CH₂Si(R¹)₃
wherein R¹ is OCH₃ or OCH₂CH₃; R is propyl; n is 1; X is CH₃O(CH₂CH₂O)ₘ; and m is an integer in a range from about 5 to about 115. More specifically, when R¹ is OCH₃, m is 6-9, coating II is 2-[methoxy(polyethyleneoxy)propyl]trimethoxysilane.

In the above examples, R was propyl for illustration, not limitation. R may comprise of at least one of an alkyl, an aryl or a combination. R¹ was OCH₃ or OCH₂CH₃ for illustration, not limitation. R¹ may comprise of at least one of an alkoxy, a hydroxyl, halide, or an alkyl, with the proviso that the three R¹ 's cannot all be an alkyl. X was CH₃O(CH₂CH₂O)ₘ for illustration and not limitation. X may independently comprise of at least one of H, amino, carboxyl, epoxy, mercapto, cyano, isocyanato, hydroxy, meth(acrylic), and a water-soluble biocompatible polymer. Furthermore, the number of X may vary as designated by n where n is an integer in a range from 1 to about 3. Each X may vary independently and are within the scope of invention.

Furthermore, the nanoparticle with coating II may be in the form of a purified single enantiomer, (S) or (R) isomer, or both.

One embodiment of the nanoparticles with coating II may have diameter of less than 50 nm. Another embodiment of the nanoparticles with coating II may have a diameter of less than 25 nm.

The number of coating structures II around the substantially monodisperse inorganic core may vary depending on the size of the substantially monodisperse inorganic core and the size of the particular coating structure II. Although the substantially monodisperse inorganic core in Scheme 2 was depicted as being surrounded by the same variation of a coating structure, the nanoparticles with coating II may comprise a plurality of variations of the coating structure II. For example, the substantially monodisperse inorganic core may be surrounded by different variations of coating structures II, as depicted above.

An aspect of the invention also encompasses modifications to the coating structures I and II. For example, nanoparticles comprising a coating with a variation of coating II is demonstrated below:
Xₙ—Y—R—Si(R¹)₃ III
wherein R independently comprises of at least one of alkyl, aryl, or combination; R¹: independently comprises of at least one of alkoxy, hydroxyl, halide, or an alkyl, with the proviso that the three R¹'s cannot all be an alkyl; n is in an integer in a range from 1 to about 3; X comprises of at least one of 0 (zero), H, amino, carboxyl, epoxy, mercapto, cyano, isocyanato, hydroxy, meth(acrylic), or a water-soluble biocompatible polymer and Y comprises 0 (zero) or an organic linkage comprising of at least one of an ether, an thioether, a disulfide, an ester, an amide, a thiourea, an urethane, or a carbamate with the proviso that when X comprises of a water soluble biocompatible polymer, Y comprises 0 or an organic linkage comprising of at least one of an ether, an thioether, a disulfide, an ester, an amide, a thiourea, an urethane, or a carbamate and when X is 0, Y is 0; and wherein the nanoparticle is substantially non-agglomerated and has a diameter in a range of about 1 nm to about 100 nm.

Coating Structure III can be prepared through typical addition or condensation reactions between functional polymers and reactive silanes each having a one of a reactive functionality such as OH, SH, NH₂, NCO, NCS, —CH═CH₂, ester, epoxide, or halide.
X-Z+Q-R—Si(R¹)₃→Xₙ—Y—R—Si(R¹)₃
wherein X is water soluble polymer; n is 1; Z is OH, SH, NH₂, NCO, NCS, —CH═CH₂, ester, epoxide, or halide; Q is OH, SH, NH₂, NCO, NCS, —CH═CH₂, ester, epoxide, or halide; Y is 0 or an organic linkage such as an ether, thioether, disulfide, ester, amide, thiourea, urethane, or carbamate; R is alkyl, aryl, or combination thereof; and R¹ is alkoxy, hydroxy or halide.

When X is a polymer such as poly(ethylene glycol) (PEG) of a specific molecular weight, especially with molecular weight higher than 400 Da or m>9, or an other polymer such as poly(propylene glycol), PNIPA, PHEMA, PVA, or peptide, the silane ligand can be synthesized from polymers and silanes with reactive groups through typical addition or condensation reactions known to the one expert in the field.

For example, X being poly(ethylene glycol)monomethyl ether amine of 5,000 Da was reacted with isocyanatopropyltrialkoxy silane, providing
CH₃O(CH₂CH₂O)ₘCH₂CH₂NH—C(O)—NH—CH₂CH₂CH₂—Si(R¹)₃
wherein X is CH₃O(CH₂CH₂O)ₘCH₂CH₂NH; n is 1; m is 6-115; Y is C(O)—NH R is CH₂CH₂CH₂; R¹ is methoxy or ethoxy.

For example, X being poly(ethylene glycol)monomethyl ether of 5,000 Da was reacted with allyl bromide and then with mercaptopropyltrialkoxy silane, providing
CH₃O(CH₂CH₂O)ₘCH₂CH₂O—CH₂CH₂CH₂—S—CH₂CH₂CH₂—Si(R¹)₃
wherein X is CH₃O(CH₂CH₂O)ₘCH₂CH₂O; n is 1; m is 15-112; Y is CH₂CH₂CH₂—S—;
R is CH₂CH₂CH₂; R¹ is methoxy or ethoxy.

### EXAMPLE 3

Alternatively novel ligands can be prepared from (III) by using polymers with reactive functionalities with known methods of coupling in the literature. For example: mPEG-NH₂ (Shearwater, Inc, molecular weight 5,000 Da) added to 3-isocyanatopropyltrimethoxysilane (Gelest, Inc) in stoichiometric amount in dry methylenechloride and stirred overnight. Product mPEG-NHC(O)NH—CH₂CH₂CH₂ (OCH₂CH₃)₃ precipitated into ether and isolated by filtration.

As an example lauric acid coated (γ—Fe₂O₃)_{1-y}(Fe₃O₄) (0.0431 mmol Fe) was mixed with 210 mg of this ligand in 5 mL toluene and sonicated 20 min and stirred overnight at room temperature. 300 μL NH₄₀H (38%) was added and sonicated at 55° C. for 5 h. Mixture stirred at room temperature overnight after addition of 2 mL isopropanol. Toluene was removed by rotary evaporator and the residue was resuspended in 10 mL milliQ water. After four hexanes wash of 10 mL each, transparent brown suspension was filtered through 100 nm filter. DLS measurements in water indicated a 25 nm diameter.

III. Physical, Magnetic, and Invivo Characterization of Nanoparticles with coating structures I and II.

The dimensions of the various nanoparticles with coatings I and II as described above were characterized using two complimentary techniques: Transmission electron microscopy (TEM) and Dynamic light scattering (DLS). Transmission electron microscopy (TEM) was used to determine the size of the inorganic core of a nanoparticle. Dynamic light scattering (DLS) or photon correlation spectroscopy (PCS) was used to determine the hydrodynamic diameter of the nanoparticles in suspension.

Nanoparticles With Coating Structures I

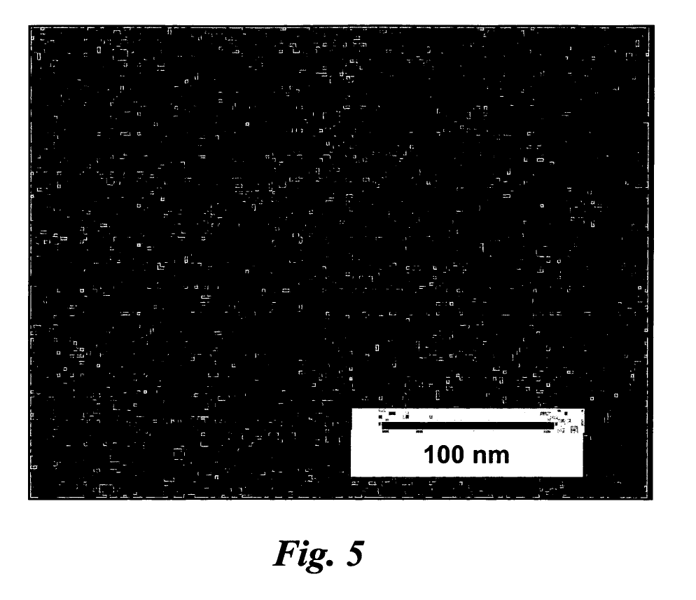

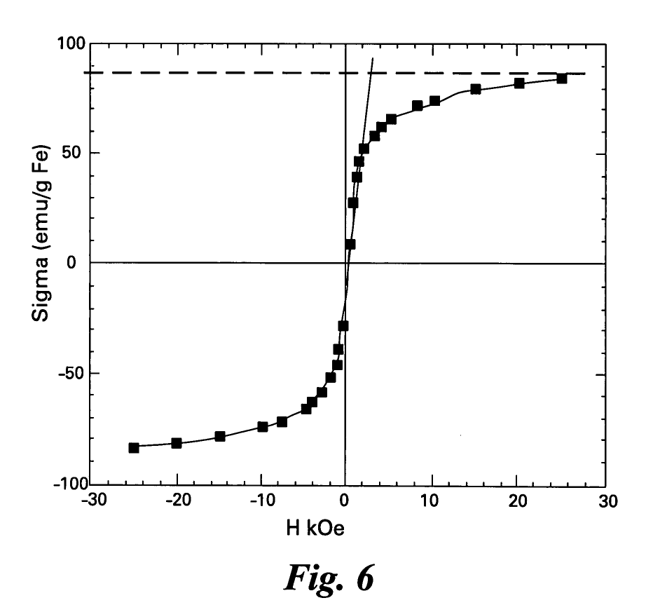

FIG. 5 shows a characteristic TEM image of iron oxide nanoparticles with coating structure I wherein R² is PEG (type: PEG-750). The nanoparticles are characterized by a high magnetic moment in presence of a magnetic field and a negligible magnetic moment in the absence of a magnetic field. Magnetization was measured using a vibrating sample magnetometer with fields up to 2,500 Gauss at 25 C. FIG. 6 shows a characteristic magnetization curve for a nanoparticle with iron oxide core and coating structure I wherein R² is PEG 750 indicating the superparamagnetic nature of the particles. The particles can have saturation magnetization in the range of 5 emu/g to 105 emu/g of metal.

As previously mentioned, MR contrast agents improve contrast by shortening the proton relaxation times more in some tissues than others and hence increasing the contrast and overall image quality. The nanoparticles were found to affect both the longitudinal relaxation (T1) and transverse relaxation times (T2). The relaxation times were measured by imaging nanoparticle suspensions at different concentrations in a GE Signa 1.5 Tesla scanner at 25° C. The nanoparticles can show relaxivities in the range: R₁ is 1˜20/mM/s and R₂ is 10-100 mM/s.

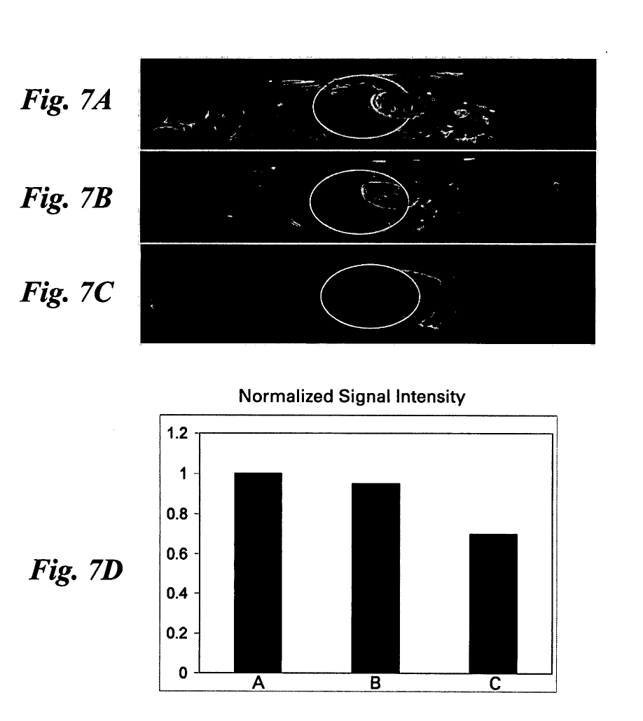

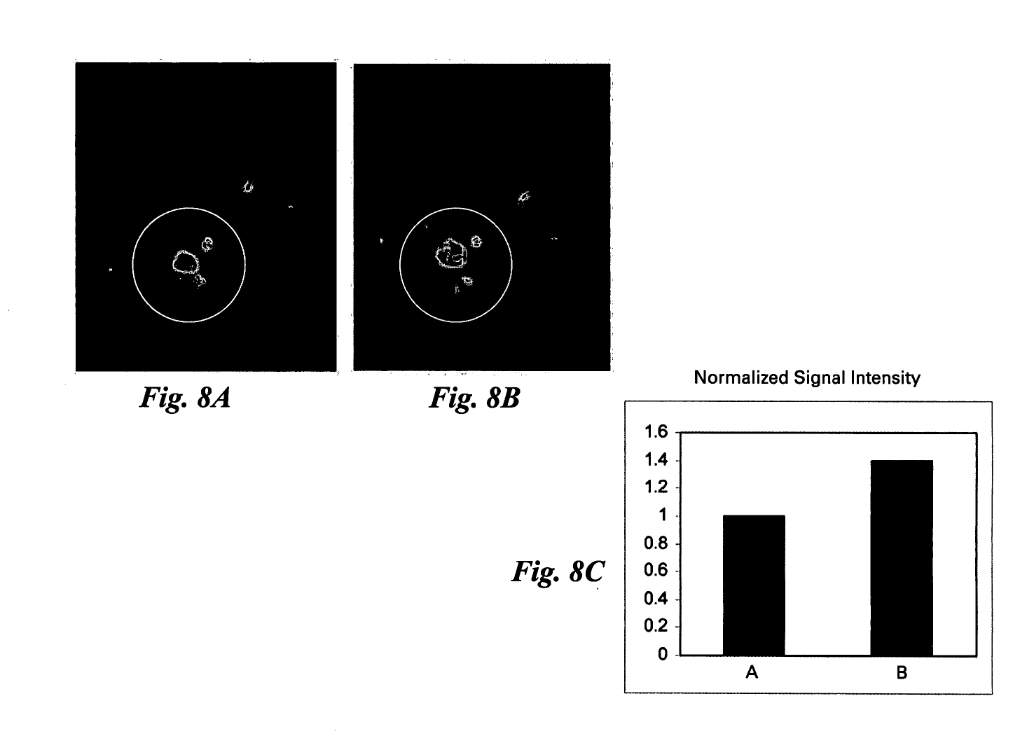

The application of nanoparticles as MR contrast agents was evaluated by performing studies on mice and rats in vivo. Rats and mice were used as the subject for illustration and not limitation; the nanoparticles are suitable for various animal species. The rats and mice were injected with known quantities of the nanoparticles and imaged using, for illustration and not limitation, GE Signa 1.5T scanner. The images before and after injection were compared to determine the effect of nanoparticles on specific tissues or organs. FIG. 7A shows a T2 weighted MR image of a mouse before injection of nanoparticles with coating structure I wherein R² is PEG-750. FIG. 7B shows a T2 weighted MR image of the same mouse 20 minutes after injection of nanoparticles with coating structure I wherein R² is PEG-750. FIG. 7C shows a T2 weighted MR image of the same mouse 24 hours after injection of nanoparticles with coating structure I wherein R² is PEG-750. While no change in the signal intensity of the liver (circled) was observed 20 minutes after injection, there was a 30% decrease in the liver signal intensity 24 hours after injection as depicted in the bar chart shown in FIG. 7D. This suggests that the nanoparticles are not rapidly taken up by the reticuloendothelial system of the liver and circulate in the blood for longer times. FIG. 8A shows T1 weighted images of the inferior vena cava (circled) of a rat before injection of nanoparticles with coating structure I wherein R² is PEG-750. FIG. 8B shows the same vena cava 10 minutes after injection of nanoparticles with coating structure I wherein R² is PEG-750. FIG. 8C shows that a 40% increase in signal intensity was observed upon injection of the nanoparticles suggesting shortening of T1 of blood due to the presence of nanoparticles.

Although dimensions of the various nanoparticles with coatings I were taken with coating I comprising R² as PEG lengths 350, 550, 750, the invention encompasses using a nanoparticle with coating I with the number of PEG chains to be 1-3 at various locations and of varying length. The number, the type, and the location of the PEG chains may vary independently and are within the scope of invention.

In the above example, the number of PEG chains for R was 3 merely for illustration. The invention encompasses the number of PEG chains to be 1-3 at various locations and of varying length. The number, the type, and the location of the PEG chains may vary independently and are within the scope of invention. Another embodiment of the nanoparticles with coating I may be wherein the R water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

For illustration and not limitation, the dimensions of nanoparticle with a coating of general formula I has been described with R² as PEG. The R² variables of nanoparticles with coating composition I may independently be any other designated R² variable. For example, R may be any water-soluble biocompatible polymer. Furthermore, the R¹, X, Y, R², n, and m groups of coating formula I may be any designated R¹, X, Y, R², n, and m groups, respectively, independent of each other. For example, when R¹ is X—COOH, R² may be any designated water-soluble biocompatible polymer. Similarly, when R² is a designated water-soluble biocompatible polymer such as PEG, R¹ may be X—COOH or any other designated R¹ variable.

An aspect of the invention also encompasses a method of improving contrast of MR image comprising administering a nanoparticle MRI contrast agent with a coating structure I to a subject in an amount that is sufficient to differentiate proton relaxation time of a tissue containing the administered nanoparticle MRI contrast agent from a background.

One embodiment, for illustration and not limitation, is when the nanoparticle contrast agent comprises of at least one of the following coating structure I: 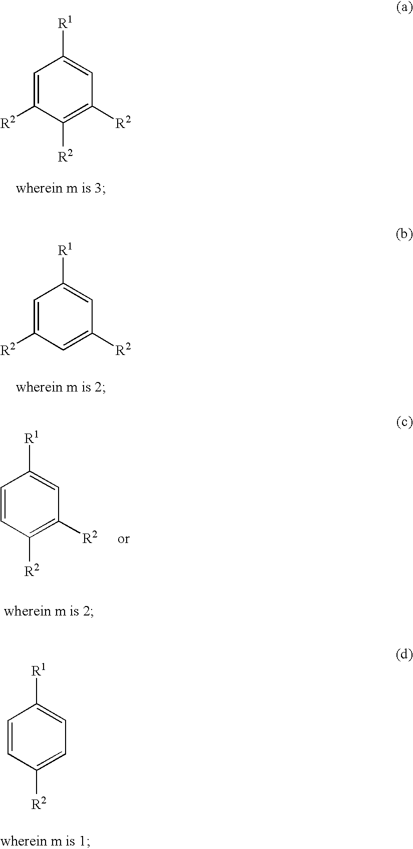
wherein R¹ is (X)ₙ—Y; wherein X is CH₂; n is an integer in a range from 0 to about 2; Y comprises of at least one of a COOH, a SO₃H, a PO₄H, a Si(OR)₃, a SiCl₃, or a NH₂; wherein R is a methyl or an ethyl; and R² independently comprises of at least one of a water-soluble biocompatible polymer. Another embodiment is when the nanoparticle contrast agent comprises of at least one of the following coating structure I: 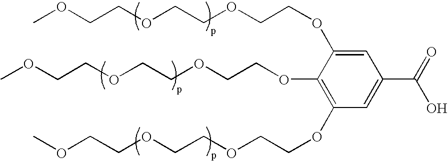
wherein m is 3; Y is COOH; X is O; n is O; R² is 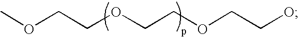
and p is an integer in a range from 5 to about 125.

An aspect of the invention also encompasses a magnetic resonance imaging contrast agent in a physiologically acceptable medium, in which the magnetic resonance imaging contrast agent comprises a population of biodegradable superparamagnetic nanoparticles with a coating structure I, wherein the nanoparticles are capable of being metabolized or excreted by a subject. One embodiment is when the nanoparticle contrast agent is capable of providing a contrast effect selected from the group consisting of a darkening effect, a brightening effect, and a combined darkening and brightening effect.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure I at a dose in a range from about 0.1 mg to about 100 mg of metal per kg of body weight; and (b) recording the MR image of the tissue or organ of the subject.

An aspect of the invention also encompasses a method for obtaining an MR image of the vascular compartment of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure I at a dose in a range from about 0.1 mg to about 100 mg of metal per kg of body weight; and (b) recording the MR image of the vascular compartment.

An aspect of the invention also encompasses a method of diagnosis comprising administering to a mammal a contrast effective amount of nanoparticle with a coating structure I suspended or dispersed in a physiologically tolerable carrier and generating a magnetic resonance image of said mammal.

Nanoparticles With Coating Structures II and III

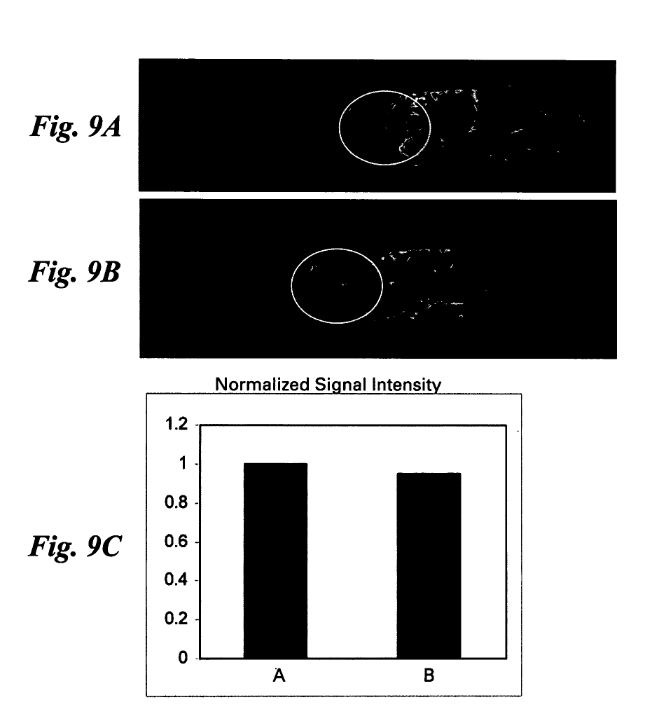

FIG. 9A shows a T2 weighted MR image of a mouse before injection of nanoparticles with a coating structure II comprising:
CH₃O(CH₂CH₂O)ₘCH₂CH₂CH₂Si(R¹)₃
(2-[methoxy(polyethyleneoxy)propyl]trimethoxysilane)
   wherein R¹ is OCH₃; R is propyl; n is 1; X is CH₃O(CH₂CH₂O)ₘ; and m is an integer in range from 6 to about 9; and wherein the nanoparticle is substantially non-agglomerated and has a diameter in a range from about 1 nm to about 30 nm.

FIG. 9B shows a T2 weighted MR image of the same mouse 20 minutes after injection of nanoparticles with a coating structure II, (2-[methoxy(polyethyleneoxy)propyl]trimethoxysilane).

FIG. 9C shows the normalized signal intensities of the liver (circled in FIGS. 9A and B) before injection (A), and 20 minutes after injection (B).

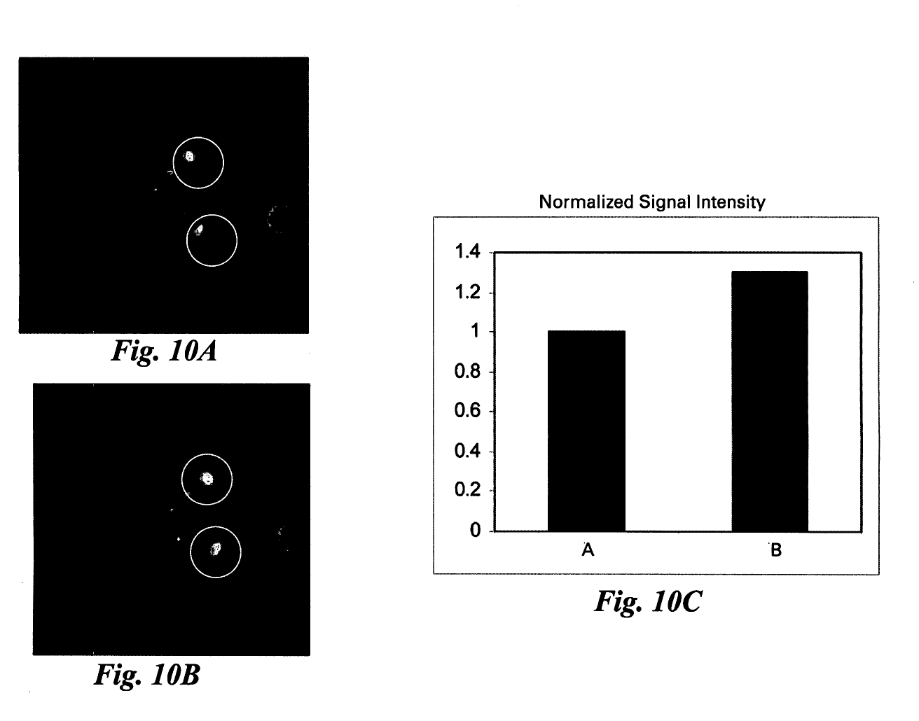

FIG. 10A shows T1 weighted images of the jugular veins (circled) of a rat before injection of nanoparticles with a coating structure II, (2-[methoxy(polyethyleneoxy)propyl]trimethoxysilane).

FIG. 10B shows the same jugular veins (circled) 10 minutes after injection of nanoparticles with a coating structure II (2-[methoxy(polyethyleneoxy)propyl]trimethoxysilane). The images indicate a brightening effect in the blood after injection of the nanoparticle contrast agent.

FIG. 10C shows the normalized signal intensities of the jugular veins (circled in FIGS. 10A and B) before injection (A) and 10 minutes after injection (B).

Although dimensions of the various nanoparticles with coatings II were taken with coating II comprising (2-[methoxy(polyethyleneoxy)propyl]trimethoxysilane, the invention encompasses using a nanoparticle with coating II wherein the number, the type, and the location of the X, R, R¹, n, and m variables vary independently as designated.

For illustration and not limitation, measurements of nanoparticle with a coating of general formula II were taken wherein the coating II comprises (2-[methoxy(polyethyleneoxy)propyl]trimethoxysilane) wherein R¹ is OCH₃ or OCH₂CH₃; R is propyl; n is 1; X is CH₃O(CH₂CH₂O)ₘ; and m is an integer in a range from about 5 to about 115. The invention encompasses measuring and using nanoparticles with a coating of general formula II wherein the X, R, R¹, n, and m variables of nanoparticle with coating composition II may independently be any designated value regardless what the other X, R, R¹, n, and m variables may be. For example, X may independently comprise of at least one of H, amino, carboxyl, epoxy, mercapto, cyano, isocyanato, hydroxy, meth(acrylic), and a water-soluble biocompatible polymer regardless of what the other R¹, Y, R², n, and m groups of general formula I may be. Similarly, when R¹ is a OCH₃ or OCH₂CH₃R¹, X may independently comprise of at least one of H, amino, carboxyl, epoxy, mercapto, cyano, isocyanato, hydroxy, meth(acrylic), and a water-soluble biocompatible polymer regardless of what R¹ is.

An aspect of the invention also encompasses a method of improving resolution of MR image comprising administering a nanoparticle MRI contrast agent with a coating structure II to a subject in an amount that is sufficient to differentiate proton relaxation time of a tissue containing the administered nanoparticle MRI contrast agent from a background. One embodiment is when the nanoparticle MRI contrast agent comprises of at least one of the following coating structure II:
CH₃O(CH₂CH₂O)ₘCH₂CH₂CH₂Si(R¹)₃
wherein R¹ is OCH₃ or OCH₂CH₃; R is propyl; n is 1; X is CH₃O(CH₂CH₂O)ₘ; and m is an integer in a range from 5 to about 115.

An aspect of the invention also encompasses a magnetic resonance imaging contrast agent in a physiologically acceptable medium, in which the magnetic resonance imaging contrast agent comprises a population of biodegradable superparamagnetic nanoparticles with coating structure II, wherein the nanoparticles are capable of being metabolized or excreted by a subject. One embodiment is when the contrast agent is capable of providing a contrast effect selected from the group consisting of a darkening effect, a brightening effect, and a combined darkening and brightening effect.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure II at a dose in a range from about 0.1 mg to about 100 mg of metal per kg of body weight; (b) recording the MR image of the tissue or organ of the subject

An aspect of the invention also encompasses a method for obtaining an MR image of the vascular compartment of an animal or a human subject comprising (a) administering to the subject, an effective amount of a magnetic resonance imaging contrast agent in a physiologically acceptable medium, wherein the magnetic resonance imaging contrast agent comprises nanoparticles with coating structure II at a dose in a range from about 0.1 mg to about 100 mg of metal per kg of body weight; and (b) recording the MR image of the vascular compartment

An aspect of the invention also encompasses a method of diagnosis comprising administering to a mammal a contrast effective of nanoparticles with coating structure II suspended or dispersed in a physiologically tolerable carrier and generating a magnetic resonance image of said mammal.

Regarding coating structure III, an aspect of the invention also encompasses a method of improving resolution of MR image comprising administering a nanoparticle MRI contrast agent with a coating structure III to a subject in an amount that is sufficient to differentiate proton relaxation time of a tissue containing the administered nanoparticle MRI contrast agent from a background.

An aspect of the invention also encompasses a magnetic resonance imaging contrast agent in a physiologically acceptable medium, in which the magnetic resonance imaging contrast agent comprises a population of biodegradable superparamagnetic nanoparticles with coating structure III, wherein the nanoparticles are capable of being metabolized or excreted by a subject. One embodiment is when the contrast agent is capable of providing a contrast effect selected from the group consisting of a darkening effect, a brightening effect, and a combined darkening and brightening effect.

An aspect of the invention also encompasses a method for obtaining an MR image of a tissue or an organ of an animal or a human subject comprising: (a) administering to the subject, an effective amount of a magnetic resonance imaging contrast agent in a physiologically acceptable medium, wherein the magnetic resonance imaging contrast agent comprises nanoparticles with coating structure III at a dose in a range from about 0.1 mg to about 100 mg of metal per kg of body weight; (b) recording the MR image of the tissue or organ of the subject

An aspect of the invention also encompasses a method for obtaining an MR image of the vascular compartment of an animal or a human subject comprising (a) administering to the subject, an effective amount of a magnetic resonance imaging contrast agent in a physiologically acceptable medium, wherein the magnetic resonance imaging contrast agent comprises nanoparticles with coating structure III at a dose in a range from about 0.1 mg to about 100 mg of metal per kg of body weight; and (b) recording the MR image of the vascular compartment

An aspect of the invention also encompasses a method of diagnosis comprising administering to a mammal a contrast effective of nanoparticles with coating structure III suspended or dispersed in a physiologically tolerable carrier and generating a magnetic resonance image of said mammal.

It will be apparent to those skilled in the art that various modifications and variations can be made in the method and system of the present invention without departing from the spirit or scope of the invention. Thus, it is intended that the present invention include modifications and variations that are within the scope of the appended claims and their equivalents.

What is claimed is:

1. A nanoparticle comprising:
a substantially monodisperse inorganic core with a surface; and
a coating substantially covering the surface of the substantially monodisperse inorganic core, wherein the coating comprises: 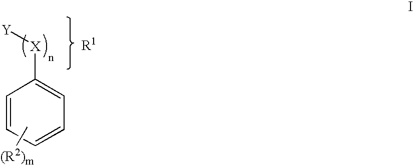
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R is methyl or ethyl;
wherein R² independently comprises of at least one of a water-soluble biocompatible polymer;
wherein m is an integer in a range from 1 to about 3; and
wherein the nanoparticle is substantially non-agglomerated and has a diameter in a range from about 1 nm to about 100 nm.

2. The nanoparticle of claim 1 wherein the coating comprises of at least one of: 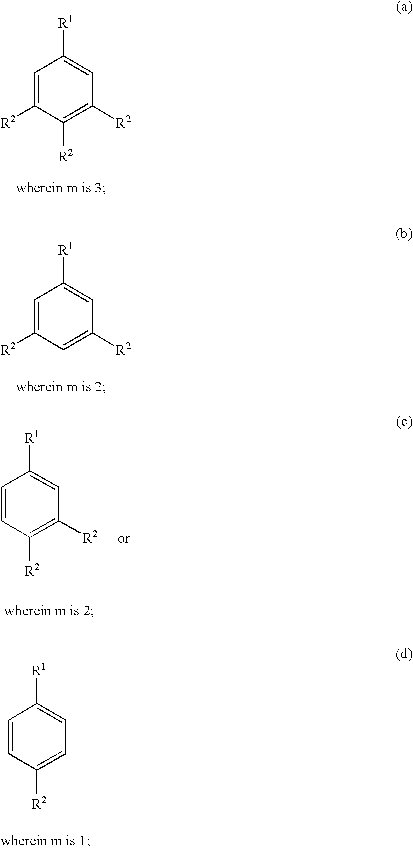
wherein m is 1;
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R is a methyl or an ethyl; and
wherein R² independently comprises of at least one of a water-soluble biocompatible polymer.

3. The nanoparticle of claim 2 wherein the coating comprises of at least one of: 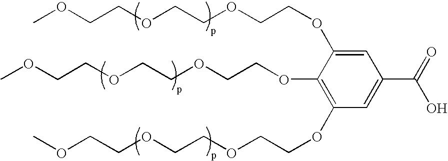
wherein m is 3; Y is COOH; X is O; n is O; R² is 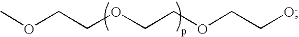
and p is an integer in a range from 5 to about 30.

4. The nanoparticle of claim 1 wherein the diameter of the nanoparticle is less than 50 nm.

5. The nanoparticle of claim 4 wherein the diameter of the nanoparticle is less than 25 nm.

6. The nanoparticle of claim 1 wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

7. The nanoparticle of claim 1 wherein the coating comprises a plurality of variations of the coating structure I.

8. A method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises: 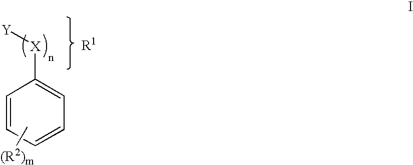
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R is a methyl or an ethyl;
wherein R² independently comprises of at least one of a water-soluble biocompatible polymer; and
wherein m is an integer in a range from 1 to about 3; the method comprising:
i) contacting the surface of the substantially monodisperse inorganic core with a 1^{st} ligand which is different from the coating structure I;
ii) adding a 2^{nd} ligand, wherein the 2^{nd} ligand is the coating structure I, in excess of an amount that is sufficient to replace the 1^{st} ligand;
iii) binding the 2^{nd} ligand on the surface of the substantially monodisperse inorganic core;
iv) providing an aqueous suspension of the substantially monodisperse inorganic core coated with the 2^{nd} ligand; and
v) removing the 1st ligand from the aqueous suspension.

9. The method of claim 8 wherein the coating comprises of at least one of: 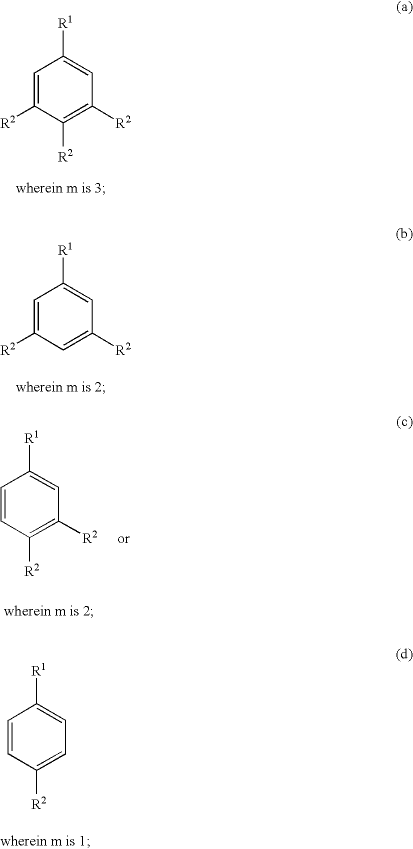
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R is a methyl or an ethyl; and
wherein R² independently comprises of at least one of a water-soluble biocompatible polymer.

10. The method of claim 9 wherein the coating comprises of least one of: 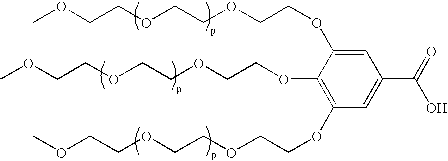
wherein m is 3; Y is COOH; X is O; n is O; R² is 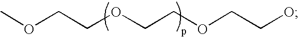
and p is an integer in a range from 5 to about 30.

11. The method of claim 8 wherein the diameter of the nanoparticle is less than 50 nm.

12. The method of claim 11 wherein the diameter of the nanoparticle is less than 25 nm.

13. The method of claim 8 wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

14. The method of claim 8 wherein the coating comprises a plurality of variations of the coating structure I.

15. A composition comprising: 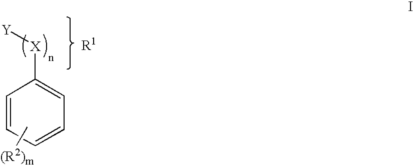
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R² independently comprises of at least one of a water-soluble biocompatible polymer;
wherein R is a methyl or an ethyl;
wherein m is an integer in a range from 1 to about 3.

16. The composition of claim 15 wherein the composition comprises of at least one of: 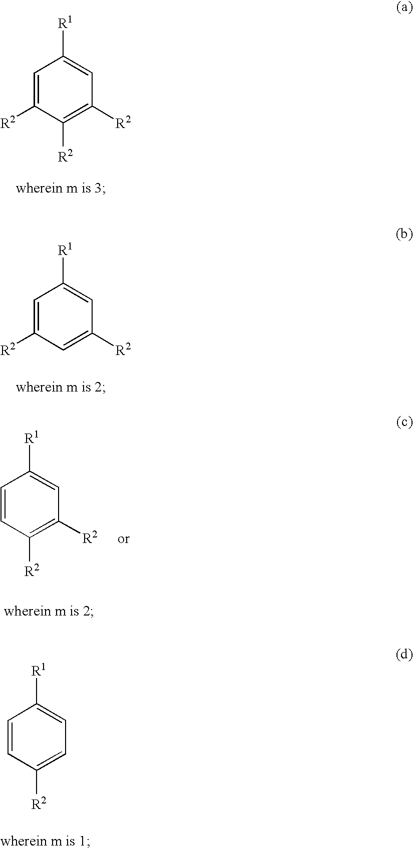
wherein R¹ is (X)ₙ—Y;
wherein X is CH₂;
wherein n is an integer in a range from 0 to about 2;
wherein R is a methyl or an ethyl;
wherein Y comprises of at least one of a COOH, a SO₃H, a PO₄H, a Si(OR)₃, a SiCl₃, or a NH₂; and
wherein R² independently comprises of at least one of a water-soluble biocompatible polymer.

17. The composition of claim 16 wherein the coating comprises of at least one of: 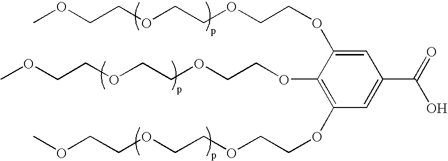
wherein m is 3; Y is COOH; X is O; n is O; R² is 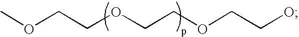
and p is an integer in a range from 5 to about 30.

18. The composition of claim 15 wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

19. The composition of claim 15 wherein the composition I comprises a plurality of variations of structure I.

20. A nanoparticle comprising:
a substantially monodisperse inorganic core; and
a coating wherein the coating substantially covering the surface of the substantially monodisperse inorganic core comprises of least one of the:
Xₙ—R—Si(R¹)₃ II
wherein R independently comprises of at least one of an alkyl, an aryl or a combination thereof;
wherein X independently comprises of at least one of H, amino, carboxyl, epoxy, mercapto, cyano, isocyanato, hydroxy, meth(acrylic), or a water-soluble biocompatible polymer;
wherein R¹ independently comprises of at least one of an alkoxy, a hydroxyl, halide, or an alkyl, with the proviso that the three R¹'s cannot all be an alkyl;
wherein n is an integer in a range from 1 to about 3; and
wherein the nanoparticle is substantially non-agglomerated and has a diameter in a range from about 1 nm to about 100 nm.

21. The nanoparticle of claim 20 wherein the R of the nanoparticle coating is C₁-8 alkyl or aryl.

22. The nanoparticle of claim 21 wherein the coating comprises of at least one of:
CH₃O(CH₂CH₂O)ₘCH₂CH₂CH₂Si(R¹)₃
wherein R¹ is OCH₃ or OCH₂CH₃
wherein R is a propyl group;
wherein n is 1;
wherein X is CH₃O(CH₂CH₂O)ₘ; and
wherein m is an integer in a range from about 5 to about 115.

23. The nanoparticle of claim 20 wherein the diameter of the nanoparticle is less than 50 nm.

24. The nanoparticle of claim 23 wherein the diameter of the nanoparticle is less than 25 nm.

25. The nanoparticle of claim 20 wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

26. The nanoparticle of claim 20 wherein the coating comprises a plurality of variations of the coating structure II.

27. A method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises:
Xₙ—R—Si(R¹)₃ II
wherein R independently comprises of at least one of an alkyl, an aryl or a combination;
wherein X independently comprises of at least one of H, amino, carboxyl, epoxy, mercapto, cyano, isocyanato, hydroxy, meth(acrylic), or a water-soluble biocompatible polymer;
wherein R¹ independently comprises of at least one of an alkoxy, a hydroxyl, halide, or an alkyl, with the proviso that the three R¹'s cannot all be an alkyl; and
wherein n is an integer in a range from 1 to about 3; the method comprising:
i) contacting the surface of the substantially monodisperse inorganic core with a 1^{st} ligand which is different from the coating structure II;
ii) adding a 2^{nd} ligand, wherein the 2^{nd} ligand is the coating structure II, in excess of an amount that is sufficient to replace the 1^{st} ligand;
iii) binding the 2^{nd} ligand on the surface of the substantially monodisperse inorganic core;
vi) providing an aqueous suspension of the substantially monodisperse inorganic core coated with the 2^{nd} ligand;
v) removing the 1st ligand from the aqueous suspension; and
vi) removing some to all of the excess 2^{nd} ligand from the aqueous suspension.

28. The method of claim 27 wherein the R of the coating is C₁₋₈ alkyl or aryl.

29. The method of claim 28 wherein the coating comprises:
CH₃O(CH₂CH₂O)ₘCH₂CH₂CH₂Si(R¹)₃
wherein R¹ is OCH₃ or OCH₂CH₃;
wherein R is a propyl group;
wherein n is 1;
wherein X is CH₃O(CH₂CH₂O)ₘ; and
wherein m is an integer in a range from about 5 to about 115.

30. The method of claim 27 wherein the diameter of the nanoparticle is less than 50 nm.

31. The method of claim 30 wherein the diameter of the nanoparticle is less than 25 nm.

32. The method of claim 27 wherein the water-soluble biocompatible polymer comprises of at least one of a polyethylene glycol, a polypropylene glycol, a poly(N-isopropylacrylamide), a poly(2-hydroxyethyl) methacrylate, a poly vinyl alcohol, a peptide, a protein, a polysaccharide, or combinations thereof.

33. The method of claim 27 wherein the coating comprises a plurality of variations of the coating structure II.

34. A method of improving resolution of MR image comprising:
administering a nanoparticle MRI contrast agent of claim 1 to a subject in an amount that is sufficient to differentiate proton relaxation time of a tissue containing the administered nanoparticle MRI contrast agent from a background.

35. The method of claim 34 wherein the nanoparticle contrast agent comprises of at least one of: 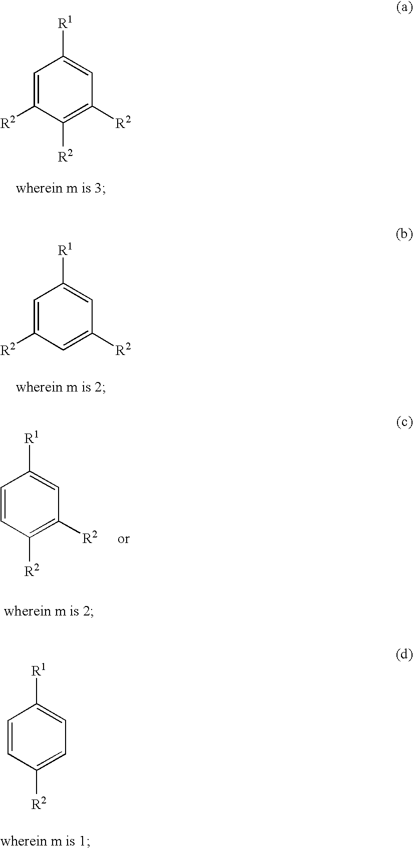
wherein R¹ is (X)ₙ—Y;
wherein X is CH₂;
wherein n is an integer in a range from 0 to about 2;
wherein Y comprises of at least one of a COOH, a SO₃H, a PO₄H, a Si(OR)₃, a SiCl₃, or a NH₂;
wherein R is a methyl or an ethyl;
wherein R² independently comprises of at least one of a water-soluble biocompatible polymer.

36. The method of claim 35 wherein the nanoparticle contrast agent comprises of at least one of: 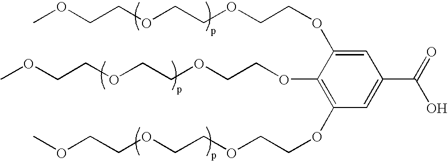
wherein m is 3; Y is COOH; X is O; n is O; R² is 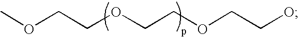
and p is an integer in a range from 5 to about 30.

37. A method of improving resolution of MR image comprising:
administering a nanoparticle MRI contrast agent of claim 20 to a subject in an amount that is sufficient to differentiate proton relaxation time of a tissue containing the administered nanoparticle MRI contrast agent from a background.

38. The method of claim 37 wherein the nanoparticle MRI contrast agent comprises of at least one of:
CH₃O(CH₂CH₂O)ₘCH₂CH₂CH₂Si(R¹)₃
wherein R¹ is OCH₃ or OCH₂CH₃;
wherein R is propyl;
wherein n is 1;
wherein X is CH₃O(CH₂CH₂O)ₘ; and
wherein m is an integer in a range from 5 to about 115.

39. A magnetic resonance imaging contrast agent in a physiologically acceptable medium, in which the magnetic resonance imaging contrast agent comprises a population of biodegradable superparamagnetic nanoparticles of claim 1, wherein said particles are capable of being metabolized or excreted by a subject.

40. The magnetic resonance imaging contrast agent of claim 39 in which said contrast agent is capable of providing a contrast effect selected from the group consisting of a darkening effect, a brightening effect, and a combined darkening and brightening effect.

41. A magnetic resonance imaging contrast agent in a physiologically acceptable medium, in which the magnetic resonance imaging contrast agent comprises a population of biodegradable superparamagnetic nanoparticles of claim 20, wherein said particles are capable of being metabolized or excreted by a subject.

42. The magnetic resonance imaging contrast agent of claim 41 in which said contrast agent is capable of providing a contrast effect selected from the group consisting of a darkening effect, a brightening effect, and a combined darkening and brightening effect.

43. A method for obtaining an MR image of a tissue or an organ of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 1 at a dose in a range from about 0.1 mg to about 100 mg of metal per kg of body weight; and
(b) recording the MR image of the tissue or organ of the subject.

44. A method for obtaining an MR image of the vascular compartment of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 1 at a dose in a range from about 0.1 mg to about 100 mg of metal per kg of body weight; and
(b) recording the MR image of the vascular compartment.

45. A method for obtaining an MR image of a tissue or an organ of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 20 at a dose in a range from about 0.1 mg to about 100 mg of metal per kg of body weight; and
(b) recording the MR image of the tissue or organ of the subject.

46. A method for obtaining an MR image of the vascular compartment of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 20 at a dose in a range from about 0.1 mg to about 100 mg of metal per kg of body weight; and
(b) recording the MR image of the vascular compartment.

47. A method of diagnosis comprising administering to a mammal a contrast effective amount of nanoparticles of claim 1 suspended or dispersed in a physiologically tolerable carrier and generating a magnetic resonance image of said mammal.

48. A method of diagnosis comprising administering to a mammal a contrast effective amount of nanoparticles of claim 20 suspended or dispersed in a physiologically tolerable carrier and generating a magnetic resonance image of said mammal.

49. A nanoparticle comprising:
a substantially monodisperse inorganic core; and
a coating substantially covering the surface of the substantially monodisperse inorganic core, wherein the coating comprises of least one of the:
Xₙ—Y—R—Si(R¹)₃ III
wherein R independently comprises of at least one of an alkyl, an aryl or a combination thereof;
wherein R¹ independently comprises of an alkoxy, a hydroxy halide, or an alkyl, with the proviso that the three R¹'s cannot all be an alkyl;
wherein n is an integer in a range of 1 to about 3; and
wherein X comprises of at least one of 0 (zero), H, amino, carboxyl, epoxy, mercapto, cyano, isocyanato, hydroxy, meth(acrylic), or a water-soluble biocompatible polymer and Y comprises 0 (zero) or an organic linkage comprising of at least one of an ether, an thioether, a disulfide, an ester, an amide, a thiourea, an urethane, or a carbamate with the proviso that when X comprises of a water soluble biocompatible polymer, Y comprises 0 or an organic linkage comprising of at least one of an ether, an thioether, a disulfide, an ester, an amide, a thiourea, an urethane, or a carbamate and when X is 0, Y is 0; and
wherein the nanoparticle is substantially non-agglomerated and has a diameter in a range of about 1 nm to about 100 nm.

50. The nanoparticle of claim 49 wherein the R of the coating is C₁-8 alkyl or aryl

51. The nanoparticle of claim 49 wherein the coating comprises of at least one of:
CH₃O(CH₂CH₂O)ₘCH₂CH₂NHC(O)NHCH₂CH₂CH₂Si(R¹)₃
wherein R¹ is OCH₃ or OCH₂CH₃
wherein R is propyl;
wherein n is 1;
wherein X is CH₃O(CH₂CH₂O)ₘ CH₂CH₂NH;
wherein m is an integer in a range from about 6 to about 115; and
wherein Y is C(O)NH.

52. A method of improving resolution of MR image comprising:
administering a nanoparticle MRI contrast agent of claim 49 to a subject in an amount that is sufficient to differentiate proton relaxation time of a tissue containing the administered nanoparticle MRI contrast agent from a background.

53. A magnetic resonance imaging contrast agent in a physiologically acceptable medium, in which the magnetic resonance imaging contrast agent comprises a population of biodegradable superparamagnetic nanoparticles of claim 49, wherein said particles are capable of being metabolized or excreted by a subject.

54. The magnetic resonance imaging contrast agent of claim 53 in which said contrast agent is capable of providing a contrast effect selected from the group consisting of a darkening effect, a brightening effect, and a combined darkening and brightening effect.

55. A method for obtaining an MR image of a tissue or an organ of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 49 at a dose in a range from about 0.1 mg to about 100 mg of metal per kg of body weight; and
(b) recording the MR image of the tissue or organ of the subject

56. A method for obtaining an MR image of the vascular compartment of an animal or a human subject comprising:
(a) administering to the subject, an effective amount of a magnetic resonance imaging contrast agent in a physiologically acceptable medium, wherein the magnetic resonance imaging contrast agent comprises the nanoparticle of claim 49 at a dose in a range from about 0.1 mg to about 100 mg of metal per kg of body weight; and
(b) recording the MR image of the vascular compartment.

57. A method of diagnosis comprising administering to a mammal a contrast effective amount of nanoparticles of claim 49 suspended or dispersed in a physiologically tolerable carrier and generating a magnetic resonance image of said mammal.

58. A method of making a substantially non-agglomerated nanoparticle having a diameter in a range from about 1 nm to about 100 nm comprising a substantially monodisperse inorganic core with a surface and a coating substantially covering the surface of the substantially monodisperse inorganic core, wherein the coating comprises:
Xₙ—Y—R—Si(R¹)₃ III
wherein R independently comprises of at least one of an alkyl, an aryl or a combination thereof;
wherein R¹ independently comprises of an alkoxy, a hydroxy halide, or an alkyl, with the proviso that the three R¹'s cannot all be an alkyl;
wherein n is an integer in a range of 1 to about 3; and
wherein X comprises of at least one of 0 (zero), H, amino, carboxyl, epoxy, mercapto, cyano, isocyanato, hydroxy, meth(acrylic), or a water-soluble biocompatible polymer and Y comprises 0 (zero) or an organic linkage comprising of at least one of an ether, an thioether, a disulfide, an ester, an amide, a thiourea, an urethane, or a carbamate with the proviso that when X comprises of a water soluble biocompatible polymer, Y comprises 0 or an organic linkage comprising of at least one of an ether, an thioether, a disulfide, an ester, an amide, a thiourea, an urethane, or a carbamate and when X is 0, Y is 0; and
wherein the nanoparticle is substantially non-agglomerated and has a diameter in a range of about 1 nm to about 100 nm.
i) contacting the surface of the substantially monodisperse inorganic core with a 1^{st} ligand which is different from the coating structure II;
ii) adding a 2^{nd} ligand, wherein the 2^{nd} ligand is the coating structure II, in excess of an amount that is sufficient to replace the 1^{st} ligand;
iii) binding the 2^{nd} ligand on the surface of the substantially monodisperse inorganic core;
vi) providing an aqueous suspension of the substantially monodisperse inorganic core coated with the 2^{nd} ligand;
v) removing the 1st ligand from the aqueous suspension; and
vi) removing some to all of the excess 2^{nd} ligand from the aqueous suspension.
